(19) United States
(12) Patent Application Publication
(10) Pub. No.: US 2026/0337669 A1
(43) Pub. Date: Oct. 29, 2026

(54) IDENTIFICATION OF SELECTIVE REGULATORS OF ONCOGENE TRANSLATION

(71) Applicant: The Regents of the University of California, Oakland, CA (US)

(72) Inventors: Joanna Kovalski, San Francisco, CA (US); Davide Ruggero, San Francisco, CA (US)

(21) Appl. No.: 19/165,956

(22) PCT Filed: Mar. 15, 2024
(86) PCT No.: PCT/US2024/020229

§ 371 Date: Sep. 16, 2025

Related U.S. Application Data
(60) Provisional application No. 63/490,953, filed on Mar. 17, 2023.

(51) Int. Cl. C12Q 1/6844; A61K 31/7105; A61P 35/00; C12N 9/22; C12N 15/113; C12Q 1/6809; C12Q 1/6811; C12Q 1/6813; C12Q 1/6869; C12Q 1/6886; C12Q 1/6897; C40B 40/06

(52) U.S. Cl. CPC C12Q 1/6844; A61K 31/7105; A61P 35/00; C12N 9/226; C12N 15/113; C12Q 1/6809; C12Q 1/6811; C12Q 1/6813; C12Q 1/6869; C12Q 1/6886; C12Q 1/6897; C40B 40/06; C12N 2310/20; C12N 2830/003; C12N 2830/34; C12Q 2600/136

(57) ABSTRACT

The present disclosure provides a bidirectional reporter system and methods to identified modulators of translation, e.g., of an oncogene or tumor suppressor gene.

### CROSS-REFERENCE TO RELATED APPLICATIONS

This application is a U.S. National Phase Application Under Section 371 of PCT/US2024/020229, filed Mar. 15, 2024, which claims priority benefit of U.S. provisional application No. 63/490,953, filed Mar. 17, 2023, which are incorporated by reference for all purposes.

### STATEMENT AS TO RIGHTS TO INVENTIONS MADE UNDER FEDERALLY SPONSORED RESEARCH AND DEVELOPMENT

This invention was made with Government support under R35 CA242986 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on Sep. 12, 2025, is named 084850-1522610-019210US_SL.xml and is 5,568 bytes in size.

### BRIEF SUMMARY

It is understood that this summary features various aspects of the disclosure and is not provided as a comprehensive summary of all embodiments encompassed by the disclosure.

In one aspect, the disclosure provides methods and compositions for identifying modulators of translation of a gene of interest, e.g., inhibitors of translation of a pro-tumorigenic gene such as an oncogene or activators of a tumor suppressor gene having decreased expression. Thus, in some embodiments, the disclosure features a bidirectional reporter nucleic acid construct for identifying regulators of oncogene or tumor suppressor gene translation, the reporter system comprising: (a) a first promoter operably linked to a first polynucleotide comprising a nucleic acid sequence encoding a first reporter molecule; and (b) a second promoter, different from the first promoter, operably linked to a second polynucleotide comprising a nucleic acid sequence encoding a second reporter molecule, wherein the second promoter and second polynucleotide are oriented in the opposite orientation relative to that of the first promoter and first polynucleotide, and wherein the second polynucleotide comprises a 5′ untranslated region from an oncogene or tumor suppressor gene at the 5′ end of the nucleic acid sequence encoding the second reporter molecule. In some embodiments, the second reporter molecule is a destabilized green fluorescent protein or firefly luciferase. In some embodiments, the second promoter is an elongation factor-1α (EF1α) promoter. In some embodiments, the first promoter is a human phosphoglycerate kinase promoter. In some embodiments, the first reporter molecule is a red fluorescent protein. In other embodiments, the first reporter molecule is destabilized mCherry or Renilla luciferase. In some embodiments, the oncogene is a RAS family member, a Myc family member, a receptor tyrosine kinase, a cell cycle polypeptide, a kinase, or a hormone receptor; or the tumor suppressor gene is a cell cycle inhibitor, Phosphatase and Tensin Homolog deleted on Chromosome 10 (PTEN) polypeptide, a polypeptide involved in DNA damage response, a Wnt signaling protein, or a p53 family member.

In a further aspect, the disclosure additionally provides a host cell, such as a cancer cell, comprising a bidirectional reporter system as described herein, e.g., in the preceding paragraph. In some embodiments, the host cell further comprises (i) CRISPRi components comprising a polynucleotide encoding a catalytically inactivated Cas protein linked to a polynucleotide encoding a transcription repressor protein operably linked to an inducible promoter, and (ii) a nucleic acid construct from a whole genome gRNA library, wherein the nucleic acid construct encodes a gRNA that targets a gene. In some embodiments, the catalytically inactivated Cas protein is dCas9, the transcription repressor is a Kruppel-associated box (KRAB) protein; and the inducible promoter is a tetracycline promoter.

Additionally, the disclosure provides a plurality of host cells comprising a population of host cells each comprising a bidirectional reporter systems as described herein. In some embodiments, the host cells of the population further comprise CRISPRi components comprising a polynucleotide encoding a catalytically inactivated Cas protein linked to a polynucleotide encoding a transcription repressor protein operably linked to an inducible promoter, and a nucleic acid construct from a whole genome gRNA library, wherein the nucleic acid encodes a gRNA that targets a gene; and wherein the population of cells comprises (i) individual host cells that each comprise a gRNA that target different genes; and (ii) at least two individual host cells that each contain a gRNA that targets the same gene, but differ in sequence from one another. In some embodiments, the catalytically inactivated Cas protein is dCas9, the transcription repressor is a Kruppel-associated box (KRAB) protein; and the inducible promoter is a tetracycline promoter.

In another aspect, the disclosure provides a method of identifying a regulator of oncogene or tumor suppressor translation, the method comprising: (a) inducing expression of the catalytically inactivated Cas protein linked to a transcription repressor protein in a plurality of cells comprising a population of cells as described herein, e.g., in the preceding paragraph; (b) detecting signal from the first reporter molecule and signal from the second reporter molecule in individual cells of the cell population to determine the ratio of signal from the first reporter molecule to signal from the second reporter molecule; (c) identifying (i) a subpopulation of cells exhibiting the highest ratio of signal from the first reporter molecule to signal from the second reporter molecule and (ii) a subpopulation of cells exhibiting the lowest ratio of signal from the first reporter molecule to signal from the second reporter molecule; (d) isolating genomic DNA from each subpopulation (c) (i) and (c) (ii); (e) amplifying sgRNA sequences in DNA from population (c) (i) and sgRNA sequences in DNA from population (c) (ii); (f) sequencing DNA from (d) to identify target genes of the sgRNA sequences present in subpopulations (c) (i) and (c) (ii); g) quantifying the sgRNAs for each target gene present in subpopulation (c) (i) relative to subpopulation (c) (ii) to identify genes that exhibit at least a two-fold statistically significant change in level in subpopulation (c) (i) compared to subpopulation (c) (ii); and (h) selecting candidate genes that are enriched in subpopulation (c) (i) as candidate translation repressors and selecting candidates genes that are enriched in subpopulation (c) (ii) as candidate translational activators. In some embodiments, (c) comprises identifying cells having at least a 2-fold change in expression of the first reporter molecule relative to the average phenotype score; and/or (c) comprises identifying cells having least a 2-fold change in expression of the second reporter molecule relative to the average phenotype score. In some embodiments, detecting step (b) comprises fluorescence activated cell sorting. In some embodiments, the population of cells is distributed to compartments of a microwell plate and detecting step (b) comprises detecting a fluorescent signal from at least one cell distributed to a microwell. In some embodiments, at least about 200 cells are distributed to each compartments of the microwell plate. In some embodiments, detecting step (b) comprises microscopy. In some embodiments, at least about 200 cells are distributed to each compartments of the microwell plate. In some embodiments, detecting step (b) comprises microscopy. In some embodiments, detecting step (b) comprises detecting a luminescent signal from one or more cells, e.g., using a microplate reader.

In addition, the disclosure provides a method of analyzing a target gene identified as described herein, e.g., in the preceding paragraph, the method comprising inhibiting expression of a gene selected in step (g) in a cell and determining the endogenous level of the oncogene or tumor suppressor protein and determining the level of endogenous RNA encoding the oncogene or tumor suppressor protein. In some embodiments, inhibiting expression comprises introducing an inhibitory RNA into the cell that targets the gene selected in step (g). In some embodiments, the inhibitory RNA is an shRNA. In some embodiments, the cell is a cancer cell. In some embodiments, the method further comprises determining the endogenous level of the oncogene or tumor suppressor mRNA in the cell. In some embodiments, the method further comprises polysome profiling to determine whether the distribution of RNA as highly translated untranslated, or translated at a low level has changed compared to a control cell in which expression of the target gene is not inhibited. In additional embodiments, the method further comprises assessing whether cells modified to express the inhibitory RNA alter anchorage-dependent growth or two-dimensional (2D) colony formation. In some embodiments, the method further assessing the cells in cell viability and/or apoptosis assays. In some embodiments, cells can be evaluated in an animal model, e.g., tumor cell xenografts in animal models such as mouse models.

In a further aspect, the disclosure provides a method of inhibiting growth of a cancer cell, the method comprising the cancer cell with an inhibitor of expression of an RNA-Binding motif 42 (RBM42) protein. In some embodiments, the inhibitor is an inhibitory RNA molecule targets an RBM42 nucleic acid sequence to inhibit expression. In some embodiments, the cancer cell is from an epithelial-type tumor or a hematological cancer.

In another aspect, the disclosure provides a method of identifying a regulator of oncogene or tumor suppressor gene translation, the method comprising: (a) incubating an individual portion of a cell population comprising individual cells each comprising a reporter construct as described herein with a plurality of candidate agents for regulating oncogene or tumor suppressor gene translation; (b) detecting signal from the first reporter molecule and signal from the second reporter molecule in individual cells of the portions to determine the ratio of signal from the first reporter molecule to signal form the second reporter molecule; (c) identifying (i) a portion of cells exhibiting the highest ratio of signal from the first reporter molecule to signal from the second reporter molecule and (ii) a portion of cells exhibiting the lowest ratio of signal from the first reporter molecule to signal from the second reporter molecule; and (c) selecting candidate genes that are enriched in portion (c) (i) as candidate translation repressors and selecting candidates genes that are enriched in portion (c) (ii) as candidate translational activators. In some embodiments, the agent is a small molecule. In some embodiments, the cell population comprises cancer cells. In some instance, a candidate agent selected by the method is further evaluated to validate the selected agent as an inhibitor or activator. In some embodiments, such evaluation comprises determining the endogenous level of oncogene or tumor suppressor mRNA and/or protein in a cell treated with the candidate agent compared to untreated control. In some embodiments, assessment of the candidate agent further comprises determining the amount of protein produced from mRNA transcripts in cells treated with the candidate agent compared to untreated control cells. In some embodiments, validating the candidate agent further comprises evaluating the ability of cells treated with the candidate agent to grow in soft agar and/or form two-dimensional colonies compared to untreated control cells; and/or evaluating cellular proliferation rates or apoptosis compared to untreated control cells.

### DESCRIPTION OF THE FIGURES

FIG. 1 provides an illustrative overview of a genetic approach to identifying selective regulators of MYC translation.

FIG. 2 illustrates a gating strategy for selective oncogene translation reporter CRISPRi screening.

FIG. 3A-D illustrates results of a CRISPRi screen and descriptions of functional roles of identified candidate regulators of MYC translation. A. Volcano plot of screen for selective mediators of MYC translation shows known and novel regulators. B. Protein-protein interaction network of top 300 candidate promoters of MYC translation. C. Molecular Function Gene Ontology enrichment of activators. D. Top 10 candidate RBP, RBM42, is increased in RNA expression in pancreatic ductal adenocarcinoma (PDA) versus healthy pancreas.

FIG. 4A-D shows validation and analysis of knockdown of an RNA binding protein (RBM42) identified in screen for regulators of MYC translation. A. Single guide validation of candidate RBM42 in the reporter CRISPRi cells compared to positive controls EIF4A1 and EIF4H. B-D. qPCR RNA quantification and Western Blot quantification of RBM42 and Myc depletion in BxPC3 pancreatic cancer cells. For the bar graphs shown in B and D, the left bar represents MYC values and the right bar represents RBM42 values in each set of bars for shCNTL, shRBM24-1 and shRBM42-2 shown on the x-axes of the graphs.

FIG. 5A-B provides illustrative data of a polysome profiling analysis to confirm effects of RBM42 knockdown on MYC mRNA translation. A. Sucrose gradient polysome profiling overview and polysome profiles with control (CNTL) or RBM42 depletion. B. qPCR of MYC and TUBB mRNA isolated from polysome profiling fractions. 1-way ANOVA ** p<. 01, ***p<. 001.

FIG. 6A-D provides data illustrating that inhibition of RBM42 represses pancreatic cancer cell growth culture, in soft agar, and in in vivo xenografts. 1-way or 2-way ANOVA **p<. 01; ***p<. 001; ****p<. 0001. Welch's t-test *p<. 05.

### DETAILED DESCRIPTION

### Terminology

As used herein, the term “pro-tumorigenic gene” broadly refers to a gene involved in tumorigenesis. A pro-tumorigenic gene is a mutated form of a gene that contributes to the development of a cancer. Mutated forms include changes in the gene copy number as well as changes in the gene sequence. In some embodiments, the pro-tumorigenic gene has a driver mutation, which refers to a somatic mutation that initiates, alone or in combination with other mutations, tumorigenesis and/or confers a replicative advantage to tumor cell. In some embodiments, a “pro-tumorigenic gene” is an oncogene in which one or more gain-of-function mutation(s) in the oncogene contribute to tumorigenesis. In some embodiments, a “pro-tumorigenic gene” refers to a tumor suppressor gene in which in which one or more partial loss-of-function mutation(s) or loss of functions mutations in the tumor suppressor gene contributes to tumorigenesis. In some embodiments, a “pro-tumorigenic gene” refers to a tumor suppressor gene that is reduced in copy number in cancer cells compared to normal counterpart cells.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. A “malignant” cancer cell typically has the potential to spread to other sites in the body (metastasize) or to invade local tissues. The term “metastasis” or a secondary tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and nonmetastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission.

The terms “polypeptide,” “peptide,” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analogs refers to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Unless otherwise indicated, a particular nucleic acid sequence also implicitly encompasses degenerate codon substitutions that encode the same amino acid (for protein encoding sequences), complementary sequences, splice variants, and the like as well as the sequence explicitly indicated. Specifically, degenerate codon substitutions may be achieved by generating sequences in which the third position of one or more selected (or all) codons is substituted with mixed-base and/or deoxyinosine residues. The term “nucleic acid” can refer to a gene, cDNA, mRNA, shRNA, siRNA, oligonucleotide, polynucleotide, and the like.

As used herein, the term “complementary” or “complementarity” refers to specific base pairing between nucleotides or nucleic acids. Base pairing may be perfectly complementary or partially complementary.

The term “gene” can refer to the segment of DNA involved in producing or encoding a polypeptide chain. It may include regions preceding and following the coding region (leader and trailer) as well as intervening sequences (introns) between individual coding segments (exons). Genes are defined by symbol and nomenclature for the human gene as assigned by the HUGO Gene Nomenclature Committee.

The term “inhibiting translation” refers to reducing the amount of protein produced from an mRNA.

To inhibit or reduce the expression of a gene in the context of gene editing or targeting RNA to inhibit expression, the sequence and/or structure of the gene may be modified such that the gene would not be transcribed (for DNA) or translated (for RNA), or would not be transcribed or translated to produce a functional protein. Various methods for inhibiting or reducing expression of a gene are described in detail further herein. “Inhibited” expression as used here refers to a decrease by at least about 30-40% or at least about 50%, or at least about 60%, or at least about 70%, or at least about 80%, or at least about 90% or up to and including a 100% decrease (i.e. absent level as compared to a reference sample). As used herein, the term “inactivated” refers to preventing expression of a polypeptide product encoded by the gene. Inactivation can occur at any stage or process of gene expression, including, but not limited to, transcription, translation, and protein expression, and inactivation can affect any gene or gene product including, but not limited to, DNA, RNA, such a mRNA, and polypeptides.

The term “inhibiting translation” refers to reducing the amount of protein produced from an mRNA. In this context, “inhibited” translation refers to a decrease by at least about 30-40% or at least about 50%, or at least about 60%, or at least about 70%, or at least about 80%, or at least about 90% or up to and including a 100% decrease (i.e. absent level as compared to a reference sample).

The term “genetic modification” as used herein refers to any modification to a cell to alter expression of a gene. Such modifications include modifications to the genome as well as modifications to introduce inhibitory sequences, such as inhibitory RNAs, into the cell.

As used herein, the phrase “modifying” in the context of modifying a genome of a cell refers to inducing a structural change in the sequence of the genome at a target genomic region. For example, the modifying can take the form of inserting a nucleotide sequence into the genome of the cell or introducing a heterologous nucleic acid into the cell. In some embodiments, a nucleotide sequence encoding a polypeptide can be altered to inactivate the gene. Such modifying can be performed, for example, by inducing a double stranded break within a target genomic region, or a pair of single stranded nicks on opposite strands and flanking the target genomic region to disrupt the gene. Methods for inducing single or double stranded breaks at or within a target genomic region include the use of a nuclease domain, e.g., Cas9, or a derivative thereof; or a base editor, e.g., a nucleotide modification domain, such as adenine deaminase, and a guide, e.g., guide RNA, directed to the target genomic region.

In the context of gene that is inhibited to modulate expression, expression of the gene may be inhibited by at least about 20%, 30%, 40%, 50%, 75%, 90% or 100%.

The term “modulate” as used with reference to modulating translation of a protein refers to increasing (e.g., activating, facilitating, enhancing, or upregulating) the amount of protein produced from an mRNA or decreasing (e.g., preventing, reducing, or downregulating) the amount of protein produced from an mRNA. In some embodiments, the amount of protein translated from an mRNA may be increased by at least about 20%, 30%, 40%, 50%, 75%, 100%, or greater compared to a control. In some embodiments, a modulator decreases the amount of protein translated from an mRNA by at least about 20%, 30%, 40%, 50%, 75%, or 90% or greater, compared to a control. In the context of a gene that is inhibited to modulate expression, expression of the gene may be inhibited by at least about 20%, 30%, 40%, 50%, 75%, 90% or 100%.

As used here, “about” refers to a range within 10% or within 20% of the indicated value.

### INTRODUCTION

The present disclosure provides a bidirectional reporter construct that can be used to evaluate translation in a population of cells comprising the reporter that are treated with candidate translation modulators. Modulators can be selected that modulate translation of a gene of interest, e.g., oncogenes, tumor suppressors, or other polypeptides of interest.

A bidirectional reporter construct employed in the methods described herein employs two different promoters that each drive expression a different reporter polypeptide, wherein the orientations of the two different promoters drive expression in the opposite direction. The nucleic acid encoding one of the reporter proteins comprises, at the 5′ end, a 5′ untranslated region sequence of a gene of interest, such as an oncogene. Reporter activity of each of the two different reporter polypeptides is measured for individual cells of a library cells in the presence or absence of candidate translational modulators as described herein to identify modulators that specifically target genes containing the 5′ untranslated region of the gene of interest.

Methods and compositions for performing such analyses, as well as illustrative embodiments, are detailed below.

### Reporter System

A bidirectional report construct of the present invention comprises two expression units that are in opposite orientations: (a) a first promoter operably linked to a polynucleotide encoding a first reporter gene that is linked to the 5′ untranslated region of an oncogene or other gene encoding a protein of interest for which it is desirable to identify modulators of expression levels; and (b) a second promoter operably linked to a polynucleotide encoding a second reporter molecule that generates a signal distinct from that of the first report molecule. The back to back opposite orientation of the expression units provide two independent mRNAs for each reporter, both of which undergo cap-dependent translation.

The reporter system provides the ability to identify agents that alter translation of the first reporter molecule; and provide the ability to assess overall translation levels in individual cells in the population. Accordingly, the reporter system can be used to identify specific modulators that target the 5′ untranslated region linked to the first reporter molecule. In typical embodiments, the reporter system employs gene knockdown technology to identify genes that when inhibited in a cell, result in modulated translation of the gene of interest.

### Promoters

Promoters selected for the reporter system are generally constitutive promoters that provide consistent levels of transcription across many cell types. The first and second promoters are typically selected so that promoter strength is largely equal, i.e., each promoter provides roughly equivalent expression. Thus, constitutive promoters such as elongation factor-1 alpha, (EF1a) promoter, phosphoglycerate kinase-1 (PGK1) promoter, beta actin promoters, e.g., human beta actin or chicken beta actin promoters or cytomegalovirus (CMV) enhancer fused to chicken beta-actin promoter, SV40 promoter, CMV immediate early promoter, human Ubiquitin C promoter (UBC), or spleen focus forming virus (SFFV) promoter.

In some embodiments, the promoter used in the construct is a bidirectional promoter, which initiates transcription in both orientations and is capable of co-regulating the expression of two genes positioned with a head-to-head orientation on opposite strands. Bidirectional promoters include histone promoters, p53/WDR79 promoter, synthetic promoters (see, e.g., Amendola et al, Nat. Biotechnol 23:108-116, 2005), and numerous other promoters identified in the human genome (see, e.g., Trinklein, et al, Genome Res. 14:62-66, 2004).

### Reporters

A reporter can be any molecule that provides a detectable signal. In some embodiments, fluorescent or luminescent reporter polypeptides are employed to simplify detection.

In some embodiments, the reporter is a luciferase reporter. Examples include firefly or Renilla Luciferase. In some embodiments, the reporter is destabilized luciferase. Such a destabilized luciferase has a shorter protein half-life compared to wildtype. Protein expression levels can then be more readily associated with changes in translational activity and increases signal-to background ratios. Destabilized versions of luciferase reporters include luciferase proteins that have been modified to include a protein degradation sequences, such as the PEST sequence, to remove cryptic regulatory sequences, and/or to remove peroxisomal targeting sequence. Illustrative luciferase proteins include luc2P, luc2CP, hRlucP, hRlucCP and NlucP).

In some embodiments, a reporter gene encodes an expressible fluorescent protein, including for example, a blue fluorescent protein (e.g. eBFP, eBFP2, Azurite, mKalamal, GFPuv, Sapphire, T-sapphire); a cyan fluorescent protein (e.g. eCFP, Cerulean, CyPet, AmCyanl, Midoriishi-Cyan, mTurquoise); a green fluorescent protein (e.g. GFP, GFP-2, tagGFP, turboGFP, EGFP, Emerald, Azami Green, Monomeric Azami Green (mAzamigreen), CopGFP, AceGFP, avGFP, ZsGreenl, Oregon Green); an orange fluorescent proteins (e.g., mOrange, mKO, Kusabira-Orange, Monomeric Kusabira-Orange, mTangerine, tdTomato, dTomato); a red fluorescent protein (e.g., mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, or Texas Red); a far red fluorescent proteins (e.g., mPlum, mNeptune); or a yellow fluorescent protein (e.g., YFP, eYFP, Citrine, SYFP2, Venus, YPet, PhiYFP, ZsYellow). In some embodiments, the fluorescent protein is a destabilized protein, such as a destabilized green fluorescent proteins.

### 5′ Untranslated Regions of Genes of Interest

A 5′ untranslated region of a gene of interest is incorporated into an expression unit of a reporter construct. The reporter for the expression unit containing the 5′ untranslated region is also referred to as the “test” reporter. The gene of interest can be any target for which it is desirable to identify modulators of translation. In some embodiments, the gene is an enzyme. In some embodiments, the gene is a protein that binds to another cellular molecules, e.g., ligands, polypeptides that participate in various protein assemblies, e.g., cellular structural proteins, channels, pores, regulatory proteins, and the like.

In some embodiments, the 5′ untranslated region is from a pro-tumorigenic gene, such as an oncogene or tumor suppressor gene, that when introduced into a cell, plays a role in inducting a cancerous phenotype in the cells, e.g., growth in soft agar; or plays a role in facilitating transition to a cancer phenotype displaying dysregulated proliferation.

In some embodiments, the 5′ untranslated region of a pro-tumorigenic gene is the 5′ untranslated region of a Ras family member, a Myc family member, a receptor tyrosine kinase, a cyclin or a cyclin-dependent kinase (CDK), a hormone receptor, a p53 family member, a DNA damage response pathway protein, a member of the phosphatidylinositol 3-kinase (PI3K) pathway, a member of the WNT signaling pathway or a serine kinase. Accordingly, in some embodiments, the pro-tumorigenic gene is Ras family member HRAS, KRAS, or NRAS; a Myc family member MYC, MYCN, MYCL; an EGFR, ERBB2, PDGFR, VEGFR, or ALK receptor tyrosine kinase, a p53 family member TP53, TP63, or TP73; a CCND1, CCNB1, CDK4 or CDK6 cyclin or CDK; a BRCA1/2, ATM, or PARP1 DNA damage response protein; an AR, ESR1, or ESR2 hormone receptor, an APC or CTNNB1 Wnt signaling pathway member, a PI3K pathway member PTEN, PIK3CA, or PI3CG, or a serine kinase such as RAF1 or BRAF.

In some embodiments, the 5′ untranslated region is from a pro-tumorigenic gene selected from the group consisting of APC, TP53, KRAS, NRAS, HRAS, PIK3CA, FAT4, LRP1 B, FBXW7, BRAF, SMAD4, PCLO, KMT2C, KMT2D, ATM, RNF213, ZFHX3, AMER1, TRRAP, ARIDIA, FATI, EP400, SOX9, RNF43, MKI67, RELN, PTPRS, PDE4DIP, CHD4, PTPRT, ANKRD11, ROBO1, MTOR, CREBBP, LRRK2, TCF7L2, KMT2B, PRKDC, UBR5, ACVR2A, ERBB4, PREX2, CARD11, NOTCH1, PTEN, NCOR2, GRIN2A, KMT2A, ATRX, CACNA1 D, ALK, MYH9, NOTCH3, POLE, BCORL1, SPEN, BCL9L, BRCA2, CUX1, ARID1 B, CTNNB1, MYH11, SMARCA4, NF1, PIK3CG, PLCG2, AXIN2, MGA, SLX4, FLT4, ERBB3, POLO, ASXL1, CAD, PTPRK, ARID2, CIC, EP300, EPHA5, NUMA1, CAMTA1, GNAS, LRP5, BCL9, PTPRD, RANBP2, IRS1, MYO5A, ROS1, IRS4, SETD1A, PIK3R1, PTPRC, COL1A1, TP53BP1, DICER1, SETBP1, ZBTB20, KDM2B, B2M, AFDN, ZNF521, and LARP4B.

### Targeted Inhibition of Genes to Identify Modulators

In some embodiments, e.g., screening for translation modulators, libraries comprising cells in which different individual genes are inhibited are employed to identify genes that can modulate translation of a target gene of interest. As used herein, the term “inhibit” includes ablating expression of the gene as well as reducing expression of the gene. A gene can be modified to inactivate the gene and/or by deleting all or part of the gene so that no functional product is produced.

In some embodiments, genes are inhibited in a library of cells comprising individual cells in which different genes are edited via a library of guide RNAs, e.g., with individual guide RNAs that can target each gene, or a subset of genes, of a genome for inhibition. In some embodiments, inhibiting expression of candidate gene(s) to create a library for screening with a reporter construct as described herein is accomplished using CRISPR/CAS methodology.

### Gene Editing to Disrupt Expression of Candidate Target Genes

In some embodiments, inhibiting expression of candidate gene to create a library for screening with a reporter construct as described herein is accomplished using CRISPRi methodology. Illustrative methods of using the CRISPR/Cas system to reduce gene expression are described in various publications, e.g., U.S. Patent Application Publication No. 2014/0170753. A CRISPR/Cas system includes a Cas protein and at least one to two ribonucleic acids that hybridize to a target motif in a target gene and direct the Cas protein to the target motif. Any CRISPR/Cas system that is capable of altering a target polynucleotide sequence to reduce or abolish expression of a gene produce in a cell can be used to generate a library in which genes are inactivated.

The Cas protein used in the invention can be a naturally occurring Cas protein or a functional derivative thereof. A “functional derivative” includes, but is not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof such as derivative Cas proteins. Suitable derivatives of a Cas polypeptide or a fragment thereof include but are not limited to mutants, fusions, covalent modifications of Cas protein or a fragment thereof.

As indicated above, in some embodiments, inhibitory CRISPR systems, e.g., CRISPRi, is employed for sequence-specific repression of gene expression of a target gene described herein. Description of CRISPRi methods is provided, e.g., in Engreitz et al., Cold Spring Harb Perspect Biol, 2019, 1 1: a035386. In some embodiments, the CRISPRi system includes a dCas9 polypeptide or a dCas12 polypeptide operably linked to a repression domain. In some embodiments, the repression domain is selected from the group consisting of a Krüppel-associated box (KRAB) repressor domain, a NuE repressor domain, a NcoR repressor domain, a SID repressor domain, a SID4X repressor domain, an EZH2 repressor domain, a FOG repressor domain, a DNMT3 A repressor domain, and a DNMT3L repressor domain.

As used throughout, a guide RNA (gRNA) sequence is a sequence that has complementarity to a target sequence in a gene and interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, publicly available whole genome libraries designed to inactive target genes (e.g., CRISPRi libraries from Addgene) or premade, or customized commercially available libraries (Genscript, Cellecta) can be employed in screening for genes that modulate translation of a gene of interest, e.g., an oncogene or tumor suppressor gene, as described herein.

### Generation of Cell Line for Screening of Translation Modulators Using an Inhibitory Gene Editing System

A reporter construct as described above is introduced into a cell line of interest, e.g., a cancer cell line, using any vectors, e.g., plasmid or viral vector, and transfection technique, e.g., electroporation, lipid-mediated delivery, biolistic particle delivery, or viral transduction. In some embodiments, viral transduction is employed e.g., using a retroviral, lentiviral, adenoviral, adeno-associated, poxvirus, herpes simplex virus, or chimeric viral vector. The reporter construct is stably expressed in the cell line of interest, e.g., using antibiotic resistance-based selection or an alternative selection system. Reporter activity of transfected or transduced cells can be assessed, e.g., via flow cytometry for fluorescent reporters, to select a population of cells that displays bright and consistent reporter signal, e.g., fluorescent signal, from each of the two reporter constructs.

An inducible inhibitory gene editing system, such as a doxycycline-inducible CRISPRi system, is also introduced into the cell line of interest. The inhibitory gene editing system is commonly introduced before the reporter constructs are introduced, but the order may vary. In some embodiments, expression of the inhibitory gene editing system can be assessed through co-induced expression of a reporter molecule, such as a fluorescent protein that generates a signal distinct from the reporters used in the report construct, to determine a transduced cell population in which most of the cells express a high level of the gene editing machinery, as well as consistent high level expression of the signals from the two components of the reporter construct.

The cell line of interest can be any cell line in which it is desirable to identify translational inhibitors, e.g., of an oncogene or tumor suppressor gene. In some embodiments, the cell line is a cancer cell line, such as a cell line from an epithelial cancer, e.g., breast, hepatocellular, colorectal, gastric, lung, prostate, pancreas, renal, bladder, or ovarian cancer. In some embodiments, the cell line is a hematological cancer cell line, such as a lymphoma, leukemia, or myeloma cell line. In some embodiments, the cell line is a neural cancer cell line such as a glioblastoma or retinoblastoma cell line; or a skin cancer cell line, such as a melanoma cell line.

### Library Screening

For screening, an sgRNA library, e.g., a lentiviral library, is introduced into the reporter cell line expressing the reporter construct and containing the inhibitory gene editing system, e.g., CRISPRi machines. In some embodiments, a whole genome sgRNA library is generated to provide multiple guides, e.g., at least two, but typically, at least three, four, or five guides per target gene. The sgRNA library is typically a viral library, e.g., a lentiviral library, in which each virus expresses one guide RNA. In some embodiments, a commercially available library, such as a lentiviral human whole genome library, may be employed. In typical embodiments, the sgRNA vector further comprises a sequence encoding a protein that generates a detectable signal, e.g., a fluorescent signal distinct from the signals generated from the two reporter proteins expressed by the two-component reporter construct, for identifying sgRNA-containing cells following introduction of the vector, e.g., typically using a viral transduction system.

In some embodiments, a smaller subset of genes, rather than a whole genome, may be evaluated for to identify candidate modulators of translation as described herein. In such instances, the sgRNA library may therefore be of a smaller size than a whole genome sgRNA library.

One of skill understands how to titrate such a library on the reporter cell line containing the inducible inhibitory gene editing machinery, e.g., CRISPRi machine, to provide an average of one guide RNA per cell. In some embodiments, e.g., when using a whole genome sgRNA library, sufficient reporter-expressing cells are infected with sgRNA virus particles to provide at least 500×, preferably at least 1000×, or greater, coverage and cells expressing sgRNA are selected using a selection system, such as an antibiotic selection system. Following selection the inducible gene editing system, e.g., CRISPRi system is induced. Any sort of inducible system may be used, but in some embodiments, the inducible gene editing system is a doxycycline inducible CRISPRi system.

Cells are then evaluated for reporter signal generated from the two components of the bidirectional reporter system as well as signal from cells that contain sgRNA. For evaluation of cells, cells are selected that have a detectable signal from the reporter molecule included in the sgRNA vector to identify cells that contain sgRNA. Cells that do not have a signal generated from the control component of the bidirectional reporter construct, i.e., the expression unit that does not contain the 5′ untranslated region of interest, are also eliminated from consideration. Signal generated by a reporter molecule can be assessed using any suitable method, e.g., flow cytometry or microscopy. The cell population is then evaluated to select a portion that exhibits the highest ratio of signal from the test reporter (from the expression unit of the bidirectional construct that includes the 5′ untranslated region of the gene of interest, e.g., oncogene) relative to the control reporter from the control expression unit of the bidirectional construct. Any number of criteria may be used for selection. In some embodiments, the top 10%, top 15%, 20%, top 25%, top 30%, or top 35% of cells with the highest ratio of signal from test reporter relative to control reporter is selected for further analysis. Similarly, a portion of cells, e.g., the bottom 15%, bottom 20%, bottom 25%, bottom 30%, or bottom 35% that exhibits the lowest ratio of expression of the test reporter to the control reporter is also selected.

Genomic DNA from the top and bottom fractions selected above is then isolated, and the sgRNA-containing sequences are amplified and sequenced, e.g., using a massively parallel sequencing technology, such as the Illumina platform, to identify sgRNAs and the genes that they target.

To prioritize candidates identified in the screen for functional validation, the fold change of each gene in the screen is evaluated. In some embodiments, candidate genes are selected that exhibit a desired statistical significance e.g., a p value of 0.05 or less across all sg RNAs that target the same gene, fold change of at least two-fold or greater in abundance between the two fractions of cells, i.e., cells with the highest ratio of signal from test reporter relative to control reporter and cells with the lowest ratio of expression of the test reporter to the control reporter. In typical embodiments, a gene that undergoes an increase in reporter signal when depleted in cells harboring sgRNAs that target the gene is selected as a candidate selective translational repressor whereas a gene that undergoes a decrease in reporter signal when depleted in cells harboring sgRNAs that target the gene is selected as a candidate selective translation activator.

### Validation of Candidates

In some embodiments, selected candidate genes are then evaluated to assess whether targeting the genes for inhibition recapitulates the screen results. For example, in some embodiments, sgRNAs that provide the greatest fold change and significance, can be individually cloned and stably expressed in the reporter-expressing cell line used for the original screen. Following induction of the inhibitory gene editing system, e.g., CRISPRi system, the ratio of reporter signaling from the bidirectional reporter component containing the 5′ untranslated region vs the control component can then be evaluated to identify candidate genes that when inhibited, resulted in a decreased ratio of translation and/or protein expression of the test component containing the 5′ untranslated regions, e.g., of an oncogene, vs. control component.

In some embodiments, e.g., when identifying translational modulators of oncogene or tumor suppressor translation, RNA expression in cancerous vs normal tissues can also be evaluated to determine genes that undergo increased or decreased mRNA expression in cancer relative to normal tissue.

In some embodiments, functional evaluation of candidate target genes for modulators of translation of the gene of interest, e.g. oncogene or tumor suppressor gene, can be performed by inhibiting expression of the candidate gene, e.g., knocking out expression, in the cell type of interest using any methodology include interfering RNA or gene editing, e.g., via CRISPR, or other nuclease editing systems such as a TALE system, zinc finger system, meganuclease system, or any gene editing system that targets a gene for editing with a nuclease. Protein levels or production of the oncogene or tumor suppressor gene can then be evaluated.

In some embodiments, mRNA and protein levels of an oncogene or tumor suppressor gene in a cell line of interest are assessed following inhibition of candidate genes using well known techniques, e.g., RT-PCR, immunoassays, and the like.

In some embodiments, e.g., when screening for modulators of translation such as modulators of oncogene gene translation, candidate target genes for further evaluation are selected that exhibit decreased protein levels or expression of the oncogene, but not significant changes in mRNA expression levels. Such modulators can be further validated using known techniques.

In some embodiments, polysome profiling using a sucrose gradient (Pringle et al., Curr Protoc Mol Biol 2019 January; 125 (1) e79) can be performed to identify the amount of mRNA that is untranslated, translated at low levels or translated at high levels to compare the distribution of mRNA from cells in which the candidate target gene is inhibited to control cells in which the candidate gene is not inhibited. A candidate target gene can then be selected that is shown to shift translation levels of the mRNA encoding the protein of interest, e.g., an oncogene, from higher levels of translation to lower levels of translation.

Candidate genes can be further tested in relevant expression systems to evaluate the effects of inhibiting the genes. For example, for candidate regulators of translation of oncogenes or tumor suppressor genes, genes can then be assessed for any number of activities including the ability to alter anchorage-dependent growth or two-dimensional (2D) colony formation and/or be evaluated in cell viability or apoptosis assays.

In some embodiments, candidate genes can be additionally be evaluated using animal models, e.g., tumor cell xenografts in mouse models.

Screening for Agents that Modulate Translation

In a further aspect, the disclosure provides a method of screening for agents that modulate translation, e.g., of an oncogene or tumor suppressor gene, using a bidirectional reporter construct as described herein.

As used herein, the term “agent” refers to any molecule, either naturally occurring or synthetic, e.g., peptide, protein, oligopeptide (e.g., from 5 to 25 amino acids in length, preferably from 10 to 20 or 12 to 18 amino acids in length), a small organic molecule (e.g., an organic molecule having a molecular weight of less than about 2500 daltons, e.g., less than 2000, less than 1000, or less than 500 daltons), circular peptide, peptidomimetic, antibody, polysaccharide, lipid, fatty acid, inhibitory RNA (e.g., siRNA or shRNA), polynucleotide, oligonucleotide, aptamer, drug compound, or other compound that modulates translation.

The reporter constructs may be introduced into any cell line in which it is desirable to perform a screening assay. For example, in some embodiments, e.g., to identify candidate modulators that influence translation of oncogenes, a reporter molecule may be introduced into a cancer cell line. The reporter molecule can be introduced into cells using any technique, including viral transduction, electroporation, ballistic delivery, lipid-mediated delivery and the like to provide a cell line that stably expresses the reporter construct.

Cells can then be treated with a candidate agent. In some embodiments, the cells are distributed into multiple portions, each of which is treated with a different candidate agent. A portion, as used herein can be one cell, but is typically more than one cell, e.g., at least about 10 cells or at least about 100 cells, at least about 500 cells, or at least about 1,000 cells. In some embodiments, a portion of cells treated with a candidate agent ranges from about 10 to about 1,000 cells.

Following treatment, cells are evaluated for signals generated from the test reporter from the test expression unit containing the 5′ untranslated region and the control reporter from the expression unit lacking the 5′ untranslated regions. Cells are eliminated from consideration that do not have a signal generated from the control component of the bidirectional reporter construct. The cell population is then evaluated to select a portion that exhibits the highest ratio of signal from the region of the bidirectional construct that includes the 5′ untranslated region of the gene of interest, e.g., oncogene, relative to the control region of the construct. In some embodiments, the top 20%, top 25%, or top 30% of cells with the highest ratio is selected for further analysis. Similarly, a portion of cells, bottom 20%, bottom 25%, bottom 30%, that exhibits the lowest ratio of expression of the test reporter to control reporter can also be selected.

To prioritize candidates identified in the screen for functional validation, the standard deviation of the signal generated from the baseline untreated control is typically calculated. In some embodiments, candidate agents are selected that exhibit a standard deviation greater than at least 1.5, typically greater than at least 2, greater than at least 3, or greater than at least 4 from the mean of the baseline signal from untreated cells. (see, e.g., Zhang et. al., J. Biomol. Screen 4:67-73, 1999).

Validation of selected candidates can be performed using assays such as those above to test the effect of the candidate agent on the amount of mRNA encoding the protein of interest that is translated compared to a control that lacks the agent, e.g., using polysome profiling. Functional assays, e.g., in instances in which oncogene 5′ untranslated regions are being tested, can also be employed to evaluate parameters associated with cell transformation, such as anchorage independent growth and 2D colony growth, as well as parameters such as cell viability and/or apoptosis. Further validation can also be performed in animal models, such as xenograft models of tumorigenesis.

### Treatment Methods

In some embodiments, a gene that plays a role in translational modulation of a gene can be a therapeutic target for treatment of a disease or condition in which it is desirable to modulate expression of the gene. For example, a subject that has cancer cells in which an oncogene has been identified, e.g., as a driver of oncogenesis, can be treated with a candidate modulator that decreases translation of the oncogene. Similarly, in instances in which a disease or conditions involves decreases in levels of a protein that contributes to the disease/condition, a subject can be treated with an agent to increase translation of the mRNA encoding the protein.

In some embodiments, a therapy can be designed to target RNA Binding Motif Protein 42 (RBM42) in cancers, e.g., epithelial cancers, hematological cancers, neuroblastoma, or any other kind of cancer in which targeting RBM42 is desirable. In some embodiments, the cancer has a myc oncogene. Illustrative protein sequences for human RBM42 isoforms are available under UniprotKB number Q9BTD8. Illustrative RBM42 nucleic acid sequence are available under the following accession numbers: HGNC: 28117; NCBI Entrez Gene: 79171; and Ensembl: ENSG00000126254.

In some embodiments in which it is desirable to decrease expression of a gene, the gene can be targeted using gene editing or by using inhibitory RNAs, such and shRNA.

In some embodiments in which it is desirable to increase expression of a gene, an expression vector, e.g., a lentiviral, adenovirus, adeno-associated virus, poxvirus, Herpes simplex virus, or “naked” plasmid that expresses the gene can be delivered to a subject.

In other embodiments, an alternative agent that modulates translation identified using a reporter molecule as described herein can be administered to a subject.

In some embodiments, a RBM42 inhibitor is administered to a subject that has cancer. In some embodiments, the cancer is an epithelial cancer such as a breast, hepatocellular, colorectal, gastric, prostate, pancreatic, or ovarian cancer. In some embodiments, the cancer is a hematological cancer such as a lymphoma, myelomas, or leukemia. In some embodiments, the cancer is a glioblastoma.

In some embodiments, a cancer having a mutation in a gene as shown in Table 1 can be treated with a translational modulator that targets the gene. In some embodiments in which it is desirable to decrease protein levels, a subject may be treated with an inhibitor such as an inhibitory RNA. In some embodiments, the inhibitor may be a small molecule. In some embodiments, the modulator targets a gene shown in Table 2. In some embodiments, the gene is UBAP2L, METTL3, or YTHDF2.

### Technique Section

This section describes the identification of RBM42, and other candidates, e.g., YTHDF2, UBAP2L, and METTL3, inhibitors of translation of MYC. The method described here serves to illustrate, not limit, the invention. One of skill understands that these techniques can be employed to identify inhibitors of translation of other genes, such as other oncogenes or tumor suppressor genes.

### Cloning/Sequence of Reporter System

To clone the 5′ untranslated region (5′UTR) of the gene of interest, e.g., oncogene, enzyme, or any other protein for which it is desirable to screen for regulators of translation, into the reporter system, first the 5′UTR was amplified via PCR. Primers were designed to provide an amplification product having a 15 bp overlap at the 5′ end with the vector and a 15 bp overlap at the 3′ end with the destabilized green fluorescent protein (dsGFP). Similarly, the dsGFP was amplified using a PCR designed to provide a 15 bp overlap at the 5′ end with the 5′UTR and a 15 bp overlap at the 3′ end with the vector. The PCR products were purified via agarose gel electrophoresis followed by column-based purification. The vector containing the phosphoglycerate kinase 1 (PGK1)-destabilized mCherry fluorescent protein (dsmCherry) in the reverse direction and the EF1a promoter in the forward direction was enzymatically digested with the restriction enzyme MluI and also gel purified.

To assemble the vector, Takara InFusion cloning was used to provide seamless junctions between the 5′UTR and the reporter protein dsGFP. Individual colonies of ampicillin-resistant transformants were selected and analyzed by Sanger sequencing to identify transformants containing the correct sequence of the reporter plasmid.

MYC 5′UTR Sequence (SEQ ID NO: 1):
aactcgctgtagtaattccagcgagaggcagagggagcgagcgggcggcc
ggctagggtggaagagccgggcgagcagagctgcgctgcgggcgtcctgg
gaagggagatccggagcgaatagggggcttcgcctctggcccagccctcc
cgctgatcccccagccagcggtccgcaacccttgccgcatccacgaaact
ttgcccatagcagcggggggcactttgcactggaacttacaacacccgag
caaggacgcgactctcccgacgcggggaggctattctgcccatttgggga
cacttccccgccgctgccaggacccgcttctctgaaaggctctccttgca
gctgcttagacgctggatttttttcgggtagtggaaaaccagcagcctcc
cgcgacg
dsGFP Sequence (SEQ ID NO: 2):
atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagaagcttagccatggcttcccgccggaggtggag
gagcaggatgatggcacgctgcccatgtcttgtgcccaggagagcgggat
ggaccgtcaccctgcagcctgtgcttctgctaggatcaatgtgtag
dsmCherry Sequence (SEQ ID NO: 3):
atggtgagcaagggcgaggaggataacatggccatcatcaaggagttcat
gcgcttcaaggtgcacatggagggctccgtgaacggccacgagttcgaga
tcgagggcgagggcgagggccgcccctacgagggcacccagaccgccaag
ctgaaggtgaccaagggtggccccctgcccttcgcctgggacatcctgtc
ccctcagttcatgtacggctccaaggcctacgtgaagcaccccgccgaca
tccccgactacttgaagctgtccttccccgagggcttcaagtgggagcgc
gtgatgaacttcgaggacggcggcgtggtgaccgtgacccaggactcctc
cctgcaggacggcgagttcatctacaaggtgaagctgcgcggcaccaact
tcccctccgacggccccgtaatgcagaagaagaccatgggctgggaggcc
tcctccgagcggatgtaccccgaggacggcgccctgaagggcgagatcaa
gcagaggctgaagctgaaggacggcggccactacgacgctgaggtcaaga
ccacctacaaggccaagaagcccgtgcagctgcccggcgcctacaacgtc
aacatcaagttggacatcacctcccacaacgaggactacaccatcgtgga
acagtacgaacgcgccgagggccgccactccaccggcggcatggacgagc
tgtacaagaagcttagccatggcttcccgccggaggtggaggagcaggat
gatggcacgctgcccatgtcttgtgcccaggagagcgggatggaccgtca
ccctgcagcctgtgcttctgctaggatcaatgtgtag

### Construction of Reporter Line

Reporter plasmids/constructs verified to have the correct 5′UTR and dsGFP sequences were transfected in combination with lentiviral packaging plasmids into HEK 293T cells to produce lentivirus. The cell supernatant containing lentivirus was used to transduce a doxycycline-inducible CRISPRi expressing (pancreatic) cancer cell line and the cells were selected using blasticidin to enrich for cells expressing the reporter system, which is linked via an IRES element to a blasticidin resistance gene. The induction of the CRISPRi system, i.e., dCas9-Krab, was tracked through the co-induced expression of a blue fluorescent protein (BFP) reporter. One of skill understand that alternative reporters can be used, e.g., alternative fluorescent reporter proteins. After 3 days of doxycycline (dox) induction, the cells are sorted using fluorescence activated cell sorting (FACS) to isolate a population of cells with the highest fluorescence of GFP, mCherry and BFP. This process was repeated until the cells were at least 95% positive for BFP when induced and exhibited a narrow, bright peak of GFP and mCherry signal.

### Functional Selective Translational Reporter CRISPRi Screen

For screening translation modulators, sgRNA library virus was generated from a commercially available (Addgene) pool of human top 5 guides (sgRNA), i.e., 5 sgRNAs per gene covering the whole genome. One of skill understand that alternative sgRNA libraries can be employed. The sgRNA plasmids contain a BFP reporter to provide the ability to track infection efficiency. HEK293T cells were transfected with the guide plasmid library along with lentiviral packaging plasmids. Following two rounds of virus collection, the virus was concentrated. This virus preparation was titrated on the cell line of interest, in this case a pancreatic cell line, to obtain a 30-40% BFP-positive population when analyzed via flow cytometry, which has been statistically determined to ensure on average 1 guide per cell.

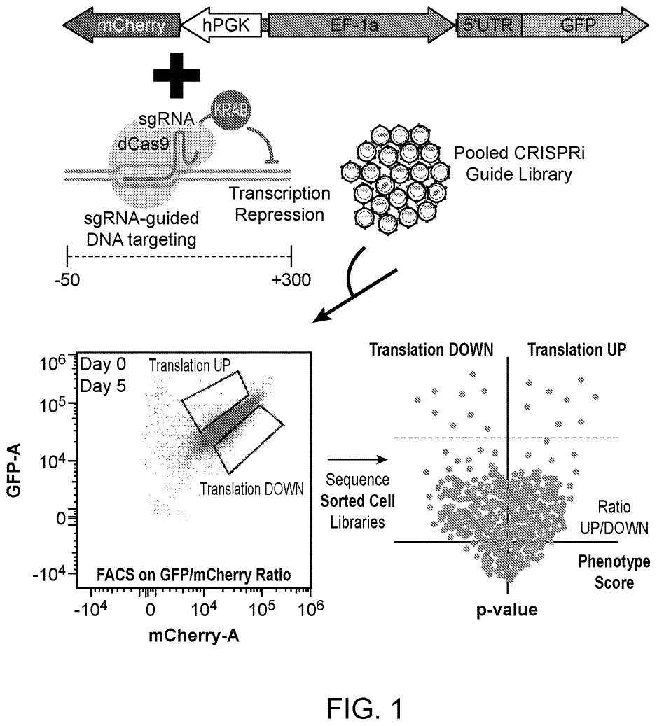

After determining an optimal ratio of virus to cell number via the above titration, reporter+CRISPRi-expressing cells were infected to ensure 1000× coverage of library in the cell population (FIG. 1). The following day the media was changed and ~48 hours after initial infection the cells were split into media containing puromycin to select for guide-expressing cells. After two days of puromycin selection, puromycin was removed from the culture media for 24 hours. After 24 hours in non-puro containing media, cells were seeded for the screen at the initiation timepoint (T₀). Sufficient cells were seeded onto 15 cm plates in dox-containing media to ensure 1000× coverage. Dox was refreshed daily and the cells were split every other day, maintaining 1000× coverage of the library.

On day 5 of doxycycline induction, the cells were harvested by brief trypsinization, quenched with media containing FBS and counted. The target number of sorted cells was based on 500× coverage total. A whole genome library with 5 guides per gene requires a minimum of 100,000,000 cells. Cells were harvested for sorting in rounds to ensure optimal cell health throughout the FACS analysis. Working in batches, cells were pelleted, strained through a 70 μM filter and resuspended in sorting buffer.

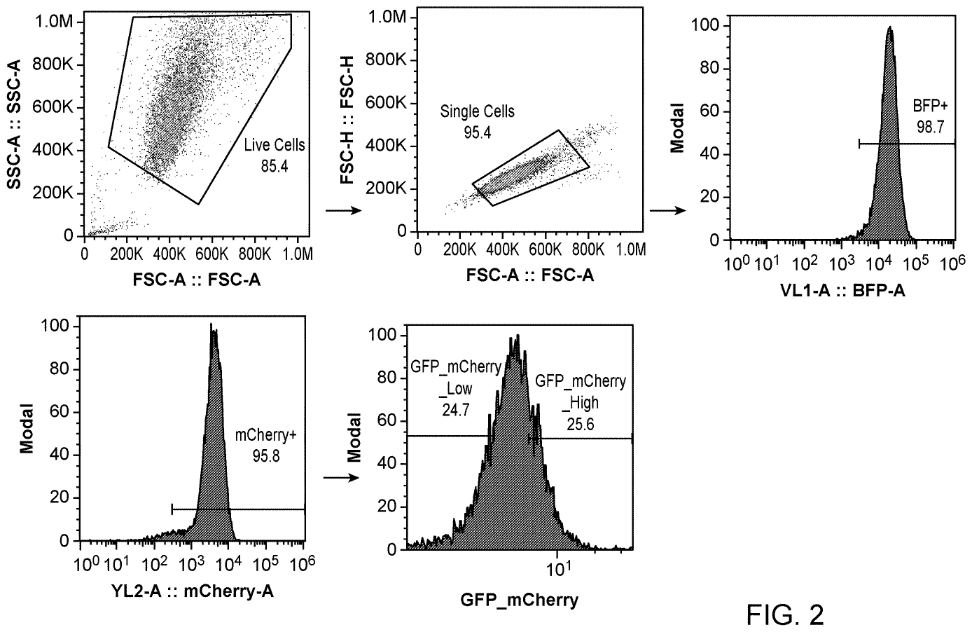

Cells were sorted using FACS with the following gating and sorting strategy (FIG. 2): (1) Gate BFP+ cells (cells with sgRNA). (2) Then, gate out any mCherry negative cells. (3) Then set gates for the top 25% and bottom 25% of the GFP/mCherry ratio. (4) Sort until at least 25,000,000 cells for each top and bottom 25%, that is at least 50,000,000 cells total equal to 500× coverage for the top 5 guide human whole genome targeted library.

Cells were pelleted and the supernatant removed. The cell pellets were snap frozen in liquid nitrogen and stored at −80C until library preparation. This process (infection of reporter/CRISPRi-expressing cells with library) was repeated for at least 2 biological replicates per cell line/reporter.

Library preparation-Genomic DNA was isolated from all cell pellets by pooling together all portions of each top or bottom 25% sample. The amount of genomic DNA for each sample was quantified and an equal amount of DNA per sample was used for PCR template, equalizing to the sample with lowest total amount. PCR primers, containing Illumina sequencing adapters and index primers for multiplexing the libraries, were used to amplify the sgRNA cassettes from the cells. All PCR reactions for a given sample were then pooled and purified using size selection beads to remove excess PCR primer and other excess PCR reaction components. Library size and concentration were analyzed for pooling and to ensure high quality sequencing, using Agilent Bioanalyzer or equivalent technology. Libraries were sequenced on an Illumina HiSeq4000 single end 50 bp with 10% PhiX spike-in to ensure library heterogeneity.

Data analyses—The sequencing data were demultiplexed based on the index added during the PCR reaction. The resulting individual FASTQ files were used as the input for publicly available CRISPRi analysis pipeline that statistically compares the abundance of guides in the top versus bottom bins for each replicate to produce a phenotype score (log 2 fold change of top 25% bin over bottom 25% bin). The analysis also generates a p-value based on comparison to the distribution of non-targeting guides as well as the consistency of the phenotype score among the 5 targeting guides per gene. This analysis provided a ranked list of genes that significantly repress the selective translation when lost (translational activators) and significantly increase selective translation when lost (translational repressors).

### Candidate Validation

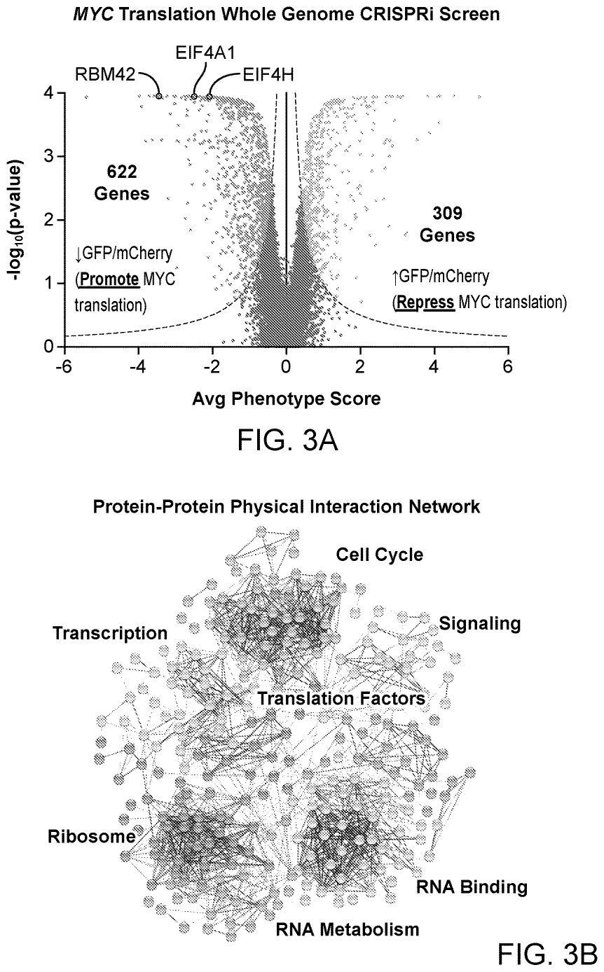

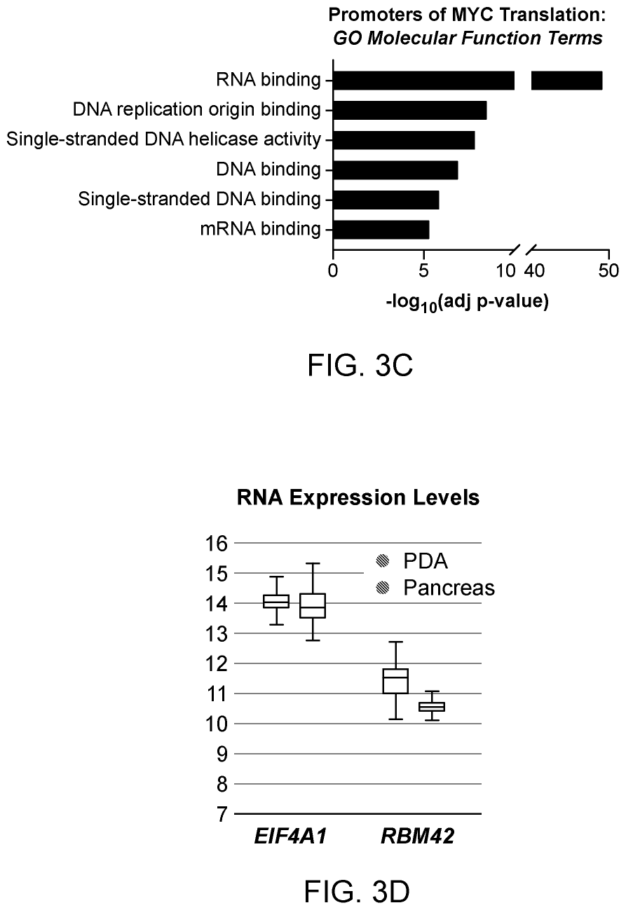

To prioritize MYC translation screen hits for downstream functional validation, the following evaluation criteria were used:
(a) The significance and fold change of each gene in the screen. Candidate genes selected for follow up had a significance (p-value)<0.05 and an absolute fold change >2. For the MYC 5′UTR we identified 622 promoters of MYC translation, including known regulators EIF4A1 and EIF4H (FIG. 3A-C).

The significant candidates were assessed for RNA expression changes between normal pancreas tissue and pancreatic cancer specimens. Genes with increased mRNA expression in cancer compared to normal were prioritized. This analysis led to the identification of the RNA Binding Protein, RBM42 (Uniprot Q9BTD8), which is in the top 10 candidate protomer genes of MYC translation and whose RNA expression was increased in pancreatic cancer versus healthy pancreas tissue (FIG. 3D).

Based on the above criteria, candidate genes were chosen for experimental validation. First, the reporter phenotype of GFP/mCherry ratio was confirmed at a single guide level. Next, functional validation was carried out, including assessment of RNA changes, protein expression changes and translation changes following candidate gene depletion using RNAi via lentiviral transduced shRNAs. RNAi was used to enable more rapid gene depletion and phenotypic evaluation.

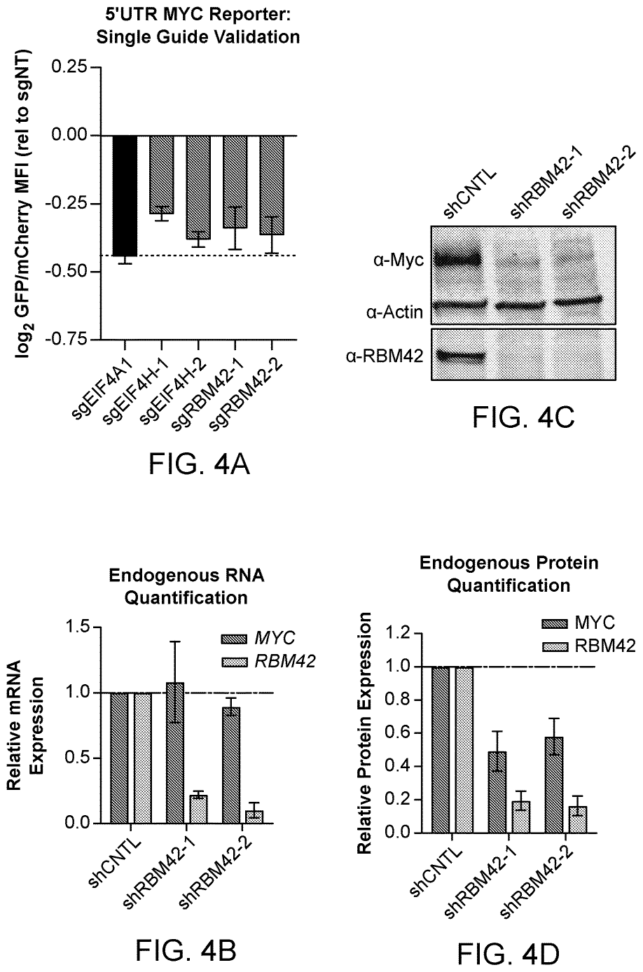

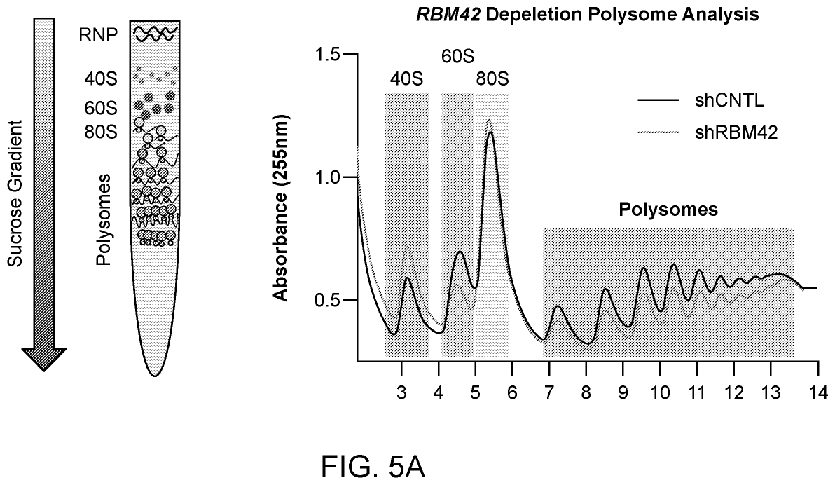

For reporter validation, the screen data were used to identify the top two scoring guides for each gene based on the phenotype score (log₂ fold change top/bottom bin guide counts) and p-value. DNA oligomers containing the targeting guide sequence and a control non-targeting guide sequence were cloned into the sgRNA vector and lentivirus was generated. The same reporter line used for the screen was infected with each sgRNA independently and selected with puromycin to ensure guide expression. Each cell line expressing the inducible CRISPRi machinery, the 5′UTR reporter system and the sgRNA was treated with dox to induce CRISPRi-induced knockdown of target genes. At day 5 of dox treatment, the cells were analyzed using flow cytometry to assay the GFP and mCherry signal. Additionally, RNA was purified from the cells and the depletion of mRNA of each target gene was confirmed via RT-qPCR. Genes were filtered by their ability to recapitulate the screen results. When genes are depleted, the GFP/mCherry ratio should decrease in accordance with whole genome CRISPRi screen results. In particular, putative activators of MYC translation were assessed for their similarity of impact on the GFP/mCherry ratio as positive controls EIF4A1 and EIF4H. This effect was observed for RBM42 (FIG. 4A).

For the genes that were validated at the single guide level as described above, RNAi reagents were generated. At least 2 independent shRNAs were cloned for each candidate regulator and lentivirus was generated. Cancer cells were infected independently with each targeting shRNA as well as control non-targeting shRNA (shCNTL). Three days after infection the cells were harvested and analyzed for endogenous oncogene RNA expression via qPCR and protein expression via western blotting. Candidates that may regulate the protein level through translation would display a dramatic change in protein expression but minimal change in RNA expression. For example, when RBM42 was depleted, Myc protein was decreased but there was minimal change in MYC mRNA expression (FIG. 4B-D).

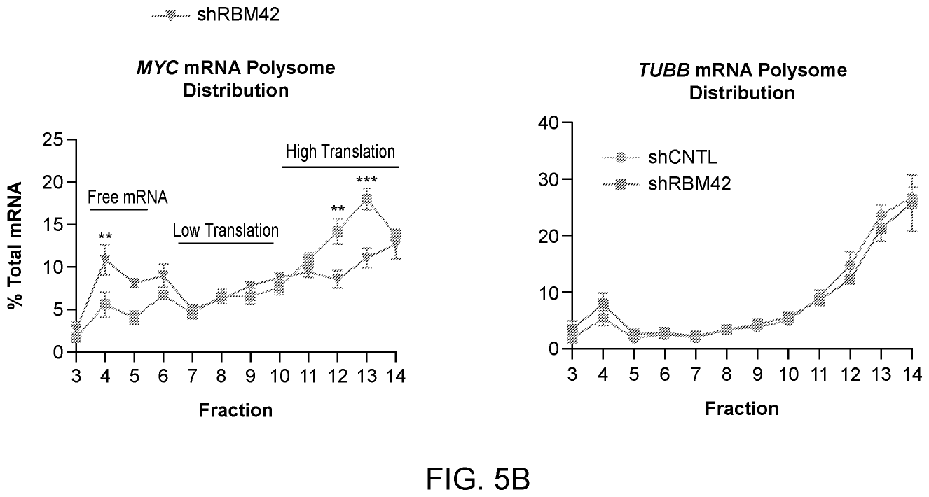

Finally, to validate that the observed change in protein expression was due to decreased translation, the gold standard translation assay, sucrose gradient polysome profiling, was used. Briefly, cells were infected with control or gene targeting shRNAs, allowed to grow for 3 days and harvested in the presence of cycloheximide to stall translation. Cells were lysed to isolate the cytoplasmic extract. Lysate was loaded onto a 10-50% sucrose gradient and ultracentrifuged to separate the sample by density. The sample was fractionated into 14 equal fractions with absorbance at 255 nm measured to visualize the RNA (FIG. 5A). To assay the translation of specific transcripts, RNA was isolated from fractions 3 through 14 and cDNA was generated. Subsequently the fractions from the control and targeted knockdown were assayed using qPCR to quantify the distribution of the target genes of interest. Specifically, RBM42 knockdown resulted in a shift of the MYC mRNA from the highly translating to the non-translating fractions, which was not observed with a control mRNA TUBB (FIG. 5B). This result supports a function for RBM42 in regulating the translation of MYC.

### Phenotypic Characterization

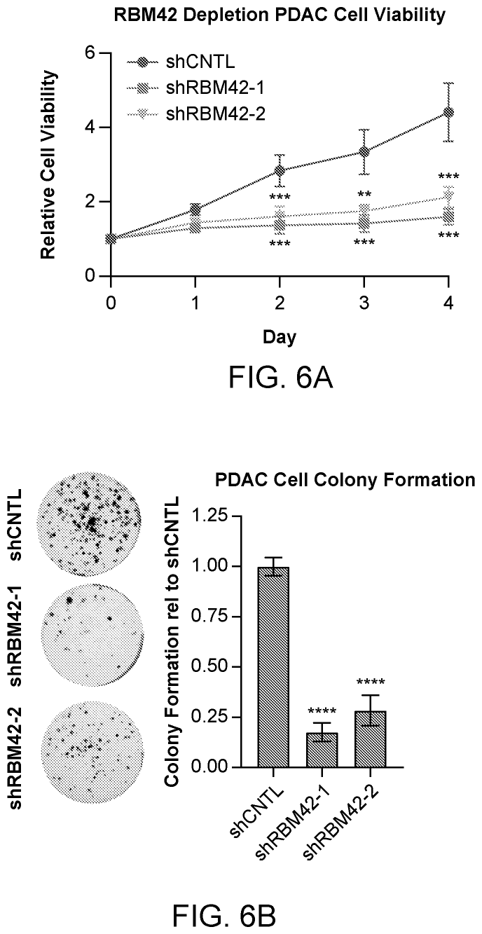

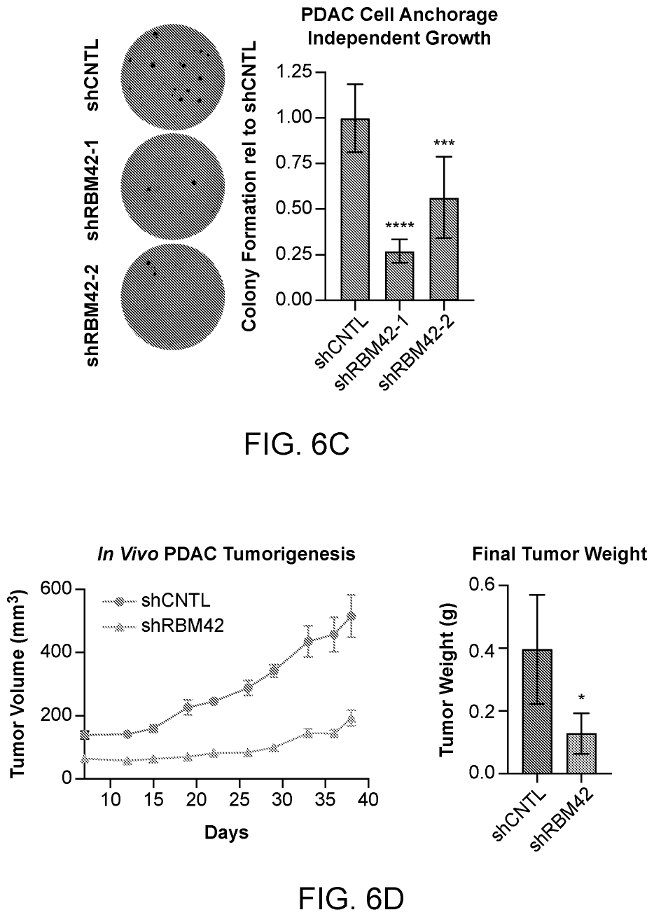

To further validate novel selective regulators of pro-oncogenic translation as bona fide dependencies of cancer cells, phenotypic characterization experiments were performed. First, a cell viability time course upon target gene depletion was conducted. Specifically, the cancer cells of interest were infected with non-targeting or shRNAs targeting the candidate regulator. The cells were selected with puromycin to enrich for cells bearing the shRNA and seeded in triplicate into 5 individual 12-well plates, for time zero (T₀) and days 1 through 4. The following morning T₀ cell viability was assayed using commercially available assays, such as Cell Titer Glo. Cell viability was measured for the following 4 days and normalized to T₀ to account for variability in cell seeding. RBM42 depletion using 2 independent shRNAs decreased pancreatic cancer cell (PDAC) viability compared to control knockdown as assessed by Cell Titer Glo signal read out on a luminometer (FIG. 6A).

Next, using the same shRNA knockdown approach, the ability of the cells to form colonies on tissue culture plates was assessed. Briefly, after puromycin selection, cells were seeded in triplicate at extremely low density in 6-well plates to ensure single colony formation. After approximately 10 days outgrowth, when the colonies were visible without magnification, the cells were stained with crystal violet. The plates were imaged, and standard image analysis software was used to calculate the percent area occupied by colonies as compared to control knockdown. RBM42 knockdown with two independent shRNAs reduced PDAC colony formation (FIG. 6B).

PDAC cells depleted for RBM42 or expressing a control shRNA were then challenged with growth in soft agar, which mimics the anchorage independent growth of cancer cells in vivo. A 0.6% agarose base was poured into 6 well plates and left to solidify at room temperature. Each cell preparation was mixed into 0.3% agarose and gently added on top of the 0.6% agarose base layer and allowed to solidify at room temperature. This was done in triplicate for each experimental replicate. As soon as the agarose solidified, the plates were moved to the tissue culture incubator. The colonies were allowed to form for 6-8 weeks with media refeeding to keep the agarose moist and to replenish nutrients. Once colonies were visible, 0.5 mL of a 5 mg/mL solution of MTT (3-(4,5-dimethylthiazol-2-yl)-2,5-diphenyltetrazolium bromide) in media was added to each well to enable visualization of the colonies that contained living cells. MTT reagent was left to incubate at 37 C for at least 1 hour or until all the colonies were pigmented. The plates were imaged and, standard image analysis software was used to count the number of colonies formed as compared to control knockdown. RBM42 knockdown with two independent shRNAs decreased the anchorage independent growth of PDAC colony formation (FIG. 6C).

### Tumorigenesis In Vivo

We also further evaluated the role of RBM42 in PDAC tumorigenesis. Examination of publicly available proteomic data of human PDAC, showed that RBM42 and Myc protein levels are significantly correlated and predictive of patient survival. In contrast, RBM42 and MYC mRNA expression are uncorrelated in the same patient cohort, supporting a translational control mechanism in vivo). To provide further clinical relevance, we examined RBM42 and Myc expression in TCGA data and human PDAC tissue. First, we stratified PDAC TCGA data to separate tumors with high and low RBM42 RNA expression and examined the enriched gene signatures in each cohort. Tumors with the highest RBM42 expression displayed significant enrichment for “MYC target” gene signatures as compared to the lower RBM42 expressing tumors. These data demonstrate that tumors with the highest RBM42 expression also possess high Myc transcriptional activity, which is indicative of high Myc protein levels. Moreover, we examined RBM42 and Myc protein expression co-occurrence in PDAC ductal structures in a panel of human PDAC tumors. The majority of ducts positive for Myc protein also had high RBM42 expression consistent with co-expression of these proteins and a role for RBM42 in regulation of Myc protein dosage. Accordingly, most ducts with low RBM42 levels were also negative for Myc protein. Together, these data indicate a strong correlation between high RBM42 and high levels of Myc protein expression, supporting a functional role of RBM42 in selective translational control in vivo in PDAC tumors.

Finally, we examined the necessity of RBM42 for pancreatic cancer tumor growth in vivo (FIG. 6D). Loss of RBM42 expression resulted in diminished tumor growth and smaller final tumor size in both MiaPaca2 and BxPC3 PDAC cancer cell xenograft models. To determine to what extent decreased Myc protein levels are responsible for reduced tumorigenesis, we constitutively expressed the Myc coding sequence in PDAC cells and tested their ability to grow in vivo following RBM42 suppression. Enforced Myc expression was sufficient to rescue BxPC3 tumor growth in vivo. These results strongly indicate that, while RBM42 controls the translation of other oncogenic factors, a predominant function of RBM42 in tumorigenesis is regulating Myc protein expression. These data substantiate that increased RBM42 activity acts as a bona fide oncogenic lesion through its selective translational control of Myc expression in human PDAC.

### Validation of Additional Candidates

Additional candidates were also selected for validations. These include UBAP2L, a multifunctional protein observed within stress granules for which RNA and protein levels are increased in cancer. UBAP2L was also shown to associate with the ribosome and promote translation, but with unknown selectivity and mechanism of action. Additionally, we validated m6A related proteins, specifically the “writer” METTL3 and “reader” YTHDF2, which emerged from our screen. m6A modification has been shown to regulate nearly all aspects of RNA metabolism from mRNA stability to transport and translation. However, target specificity and the impact on translation efficiency of certain m6A writers and readers are highly context dependent and only beginning to be unraveled in cancer. Decreased expression of UBAP2L, METTL3 and YTHDF2 in pancreatic ductal adenocarcinoma cells (PDACs) expressing the fluorescence reporter system described above demonstrated a selective decrease in the MYC translational reporter compared to control. This selective change in the MYC translation reporter was not observed for a panel of related RBPs that were not enriched in the functional CRISPRi screen. Next, we interrogated whether these candidate regulators selectively altered endogenous Myc protein dosage. Short term depletion of UBAP2L, METTL3 or YTHDF2 in PDAC cells resulted in decreased Myc protein expression without substantial changes in MYC mRNA levels, implicating a translational control mechanism. To formally show that MYC mRNA translation was decreased with the loss of function of these hits, we employed sucrose gradient fractionation polysome profiling, as described above. In these experiments, we observed a shift of the MYC mRNA from the polysomes (highly translating fractions) to the untranslated fractions, when the expression of UBAP2L, METTL3, or YTHDF2 was reduced. The translational control of housekeeping genes was not altered in these conditions. UBAP2L is significantly correlated with Myc protein expression levels in human PDAC quantitative proteomics (Spearman's correlation=0.483; p-value=. 0496), which further supports the human clinical relevance of these MYC translational regulators.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference for the purpose for which it was cited to the same extent as if each reference was individually incorporated by reference.

TABLE 1

| | | average | |
|---|---|---|---|
| | Mann-Whitney | phenotype of | |
| Gene | p-value | strongest 3 | DS |
| CUL2 | 0.0001 | −5.407 | −21.263 |
| MAGOH | 0.0001 | −3.971 | −15.689 |
| SRSF3 | 0.0002 | −3.917 | −14.828 |
| SEC61A1 | 0.0001 | −3.699 | −14.620 |
| CTR9 | 0.0001 | −3.574 | −14.121 |
| YTHDC1 | 0.0001 | −3.492 | −13.795 |
| RBM42 | 0.0001 | −3.441 | −13.595 |
| CBLL1 | 0.0001 | −3.413 | −13.485 |
| CNOT3 | 0.0001 | −3.344 | −13.100 |
| INTS3 | 0.0001 | −3.226 | −12.745 |
| MAGOHB | 0.0006 | −3.798 | −12.309 |
| SAE1 | 0.0001 | −3.041 | −12.000 |
| CHAF1B | 0.0001 | −3.003 | −11.852 |
| RRM1 | 0.0005 | −3.622 | −11.807 |
| IPO13 | 0.0001 | −2.989 | −11.806 |
| CENPE | 0.0001 | −3.014 | −11.540 |
| GON4L | 0.0002 | −3.133 | −11.502 |
| POLRID | 0.0001 | −2.833 | −11.119 |
| CASP8AP2 | 0.0005 | −3.370 | −10.984 |
| POLE | 0.0001 | −2.744 | −10.830 |
| CLSPN | 0.0001 | −2.690 | −10.620 |
| WDR12 | 0.0001 | −2.718 | −10.591 |
| MCM10 | 0.0001 | −2.652 | −10.469 |
| RFC4 | 0.0001 | −2.643 | −10.429 |
| SSRP1 | 0.0001 | −2.625 | −10.367 |
| VHL | 0.0003 | −2.916 | −10.178 |
| USP39 | 0.0001 | −2.557 | −10.092 |
| RBM12 | 0.0001 | −2.560 | −10.078 |
| CPNE1 | 0.0001 | −2.560 | −10.063 |
| RRM2 | 0.0001 | −2.552 | −10.058 |
| ZC3H13 | 0.0001 | −2.572 | −10.037 |
| SUPT6H | 0.0001 | −2.541 | −10.000 |
| CSTF1 | 0.0002 | −2.675 | −9.980 |
| PRIM1 | 0.0001 | −2.527 | −9.957 |
| SEC13 | 0.0001 | −2.532 | −9.955 |
| EIF4A1 | 0.0001 | −2.485 | −9.802 |
| SAP18 | 0.0002 | −2.686 | −9.792 |
| PA2G4 | 0.0001 | −2.452 | −9.681 |
| TBP | 0.0001 | −2.485 | −9.581 |
| TONSL | 0.0006 | −2.949 | −9.571 |
| PAF1 | 0.0006 | −2.904 | −9.393 |
| EEF1A1 | 0.0001 | −2.354 | −9.291 |
| CCT6A | 0.0001 | −2.279 | −8.974 |
| CXXC1 | 0.0001 | −2.309 | −8.972 |
| GTF3C2 | 0.0001 | −2.278 | −8.907 |
| PRPF38B | 0.0001 | −2.255 | −8.859 |
| EIF4A3 | 0.0002 | −2.348 | −8.763 |
| CDC6 | 0.0002 | −2.264 | −8.604 |
| INTS4 | 0.0006 | −2.633 | −8.579 |
| PRPF4B | 0.0001 | −2.203 | −8.572 |
| DDX56 | 0.0001 | −2.174 | −8.551 |
| TERF2 | 0.0007 | −2.703 | −8.525 |
| SUPT16H | 0.0001 | −2.146 | −8.441 |
| CSDE1 | 0.0001 | −2.132 | −8.401 |
| RBMX | 0.0001 | −2.130 | −8.388 |
| SLBP | 0.0001 | −2.125 | −8.349 |
| PSMD13 | 0.0005 | −2.476 | −8.269 |
| GINS1 | 0.0001 | −2.090 | −8.239 |
| ERH | 0.0001 | −2.117 | −8.227 |
| EIF4H | 0.0001 | −2.071 | −8.169 |
| NIP7 | 0.0001 | −2.083 | −8.148 |
| MCM2 | 0.0005 | −2.477 | −8.073 |
| CHAF1A | 0.0001 | −2.076 | −8.058 |
| MCM5 | 0.0001 | −2.024 | −7.976 |
| SPEN | 0.0001 | −2.027 | −7.948 |
| ZNF574 | 0.0001 | −1.998 | −7.860 |
| GTF3C3 | 0.0004 | −2.290 | −7.782 |
| RTF1 | 0.0011 | −2.641 | −7.777 |
| UMPS | 0.0001 | −1.953 | −7.686 |
| INTS9 | 0.0002 | −2.092 | −7.652 |
| POLR3A | 0.0001 | −1.938 | −7.606 |
| NPAT | 0.0003 | −2.136 | −7.600 |
| GNL2 | 0.0001 | −1.925 | −7.589 |
| POLR1C | 0.0013 | −2.601 | −7.468 |
| RPL4 | 0.0006 | −2.328 | −7.449 |
| OGT | 0.0001 | −1.896 | −7.448 |
| GTF3C1 | 0.0046 | −3.189 | −7.446 |
| NRBP1 | 0.0004 | −2.158 | −7.342 |
| RAD51 | 0.0001 | −1.866 | −7.337 |
| CCAR1 | 0.0001 | −1.866 | −7.331 |
| CCT8 | 0.0001 | −1.865 | −7.253 |
| CEP192 | 0.0001 | −1.851 | −7.245 |
| RPA1 | 0.0006 | −2.255 | −7.239 |
| NOP2 | 0.0001 | −1.840 | −7.205 |
| PPAN | 0.0001 | −1.836 | −7.203 |
| TICRR | 0.0006 | −2.207 | −7.165 |
| CCT2 | 0.0001 | −1.816 | −7.138 |
| HSPA8 | 0.0002 | −1.866 | −7.119 |
| MCM3 | 0.0001 | −1.814 | −6.992 |
| RSL1D1 | 0.0001 | −1.757 | −6.826 |
| MYBBP1A | 0.0001 | −1.752 | −6.824 |
| MRGBP | 0.0001 | −1.738 | −6.813 |
| PPAN-P2RY11 | 0.0006 | −2.094 | −6.805 |
| KMT2E | 0.0002 | −1.809 | −6.791 |
| RPF2 | 0.0001 | −1.745 | −6.789 |
| POLD2 | 0.0001 | −1.721 | −6.762 |
| BDP1 | 0.0002 | −1.784 | −6.718 |
| SNRPF | 0.0002 | −1.758 | −6.696 |
| PRIM2 | 0.0006 | −2.063 | −6.668 |
| ORC1 | 0.0009 | −2.176 | −6.645 |
| NVL | 0.0001 | −1.714 | −6.638 |
| EXOSC8 | 0.0001 | −1.710 | −6.597 |
| BRF1 | 0.0001 | −1.671 | −6.501 |
| LSM6 | 0.0028 | −2.541 | −6.497 |
| SURF6 | 0.0001 | −1.667 | −6.451 |
| SRSF7 | 0.0007 | −2.053 | −6.450 |
| POLR3D | 0.0001 | −1.654 | −6.432 |
| GTF3C6 | 0.0002 | −1.745 | −6.377 |
| HEATR1 | 0.0001 | −1.627 | −6.369 |
| AURKA | 0.0007 | −2.026 | −6.358 |
| PSMD4 | 0.0001 | −1.621 | −6.320 |
| RRS1 | 0.0001 | −1.640 | −6.311 |
| KAT8 | 0.0007 | −2.002 | −6.301 |
| POLR1E | 0.0001 | −1.608 | −6.283 |
| MCM6 | 0.0001 | −1.597 | −6.254 |
| SNRPC | 0.0005 | −1.889 | −6.252 |
| EXOSC5 | 0.0001 | −1.595 | −6.240 |
| RBM33 | 0.0001 | −1.590 | −6.192 |
| UBA2 | 0.0002 | −1.658 | −6.191 |
| NCL | 0.0002 | −1.673 | −6.188 |
| CAD | 0.0001 | −1.585 | −6.162 |
| NUP160 | 0.0007 | −1.954 | −6.135 |
| RAD9A | 0.0001 | −1.576 | −6.130 |
| CDC7 | 0.0002 | −1.625 | −6.119 |
| MCM3AP | 0.0003 | −1.699 | −6.115 |
| CRCP | 0.0002 | −1.647 | −6.088 |
| RFC3 | 0.0001 | −1.553 | −6.083 |
| TFPT | 0.0001 | −1.545 | −6.036 |
| NCBP1 | 0.0054 | −2.663 | −6.029 |
| CCT4 | 0.0001 | −1.535 | −6.010 |
| IPO7 | 0.0001 | −1.545 | −5.981 |
| ATP6V1H | 0.0005 | −1.784 | −5.944 |
| SOCS3 | 0.0006 | −1.828 | −5.940 |
| CDC73 | 0.0017 | −2.146 | −5.938 |
| CNOT2 | 0.0006 | −1.850 | −5.938 |
| RPL12 | 0.0002 | −1.610 | −5.934 |
| SDAD1 | 0.0001 | −1.513 | −5.889 |
| MAK16 | 0.0001 | −1.505 | −5.886 |
| SPC25 | 0.0002 | −1.558 | −5.862 |
| RBM28 | 0.0001 | −1.475 | −5.758 |
| INTS6 | 0.0001 | −1.493 | −5.747 |
| KIAA1429 | 0.0007 | −1.833 | −5.739 |
| DKC1 | 0.0002 | −1.489 | −5.686 |
| DROSHA | 0.0002 | −1.568 | −5.666 |
| MCM4 | 0.0002 | −1.538 | −5.665 |
| RPA2 | 0.0028 | −2.214 | −5.662 |
| VARS | 0.0001 | −1.442 | −5.630 |
| SS18L2 | 0.0006 | −1.734 | −5.620 |
| CD3EAP | 0.0001 | −1.465 | −5.613 |
| ZC3H8 | 0.0002 | −1.485 | −5.600 |
| RFC2 | 0.0004 | −1.625 | −5.585 |
| GINS4 | 0.0001 | −1.443 | −5.580 |
| KIF11 | 0.0002 | −1.536 | −5.578 |
| RPA3 | 0.0001 | −1.448 | −5.549 |
| SRP14 | 0.0002 | −1.540 | −5.548 |
| BORA | 0.0002 | −1.535 | −5.540 |
| STIP1 | 0.0001 | −1.424 | −5.529 |
| LSM5 | 0.0010 | −1.848 | −5.527 |
| LEO1 | 0.0095 | −2.725 | −5.509 |
| POLD3 | 0.0010 | −1.830 | −5.473 |
| MDN1 | 0.0001 | −1.411 | −5.435 |
| EIF6 | 0.0006 | −1.671 | −5.420 |
| SMC4 | 0.0003 | −1.500 | −5.400 |
| ILF2 | 0.0001 | −1.407 | −5.397 |
| TIPIN | 0.0002 | −1.446 | −5.359 |
| HINFP | 0.0006 | −1.646 | −5.349 |
| ELAVL1 | 0.0002 | −1.479 | −5.343 |
| BRD8 | 0.0005 | −1.627 | −5.342 |
| TAF10 | 0.0003 | −1.529 | −5.326 |
| SMC1A | 0.0001 | −1.376 | −5.305 |
| RPF1 | 0.0002 | −1.454 | −5.303 |
| LSM4 | 0.0004 | −1.548 | −5.287 |
| PGGT1B | 0.0001 | −1.374 | −5.287 |
| WDR75 | 0.0006 | −1.626 | −5.271 |
| SENP6 | 0.0003 | −1.491 | −5.256 |
| NOL8 | 0.0013 | −1.817 | −5.255 |
| ZNF335 | 0.0002 | −1.454 | −5.250 |
| DCTN6 | 0.0002 | −1.426 | −5.240 |
| ZNRD1 | 0.0001 | −1.358 | −5.233 |
| RRP1 | 0.0002 | −1.437 | −5.217 |
| ATP6V0C | 0.0002 | −1.421 | −5.215 |
| DNAJB8 | 0.0023 | −1.972 | −5.215 |
| UBAP2L | 0.0001 | −1.336 | −5.166 |
| RPL7 | 0.0006 | −1.607 | −5.160 |
| ATP6V1B2 | 0.0001 | −1.325 | −5.159 |
| TAF4 | 0.0001 | −1.325 | −5.152 |
| MSL1 | 0.0009 | −1.679 | −5.140 |
| SRBD1 | 0.0001 | −1.310 | −5.100 |
| MBNL1 | 0.0002 | −1.335 | −5.099 |
| PRPF6 | 0.0001 | −1.319 | −5.095 |
| TEX10 | 0.0006 | −1.577 | −5.089 |
| GINS2 | 0.0002 | −1.340 | −5.075 |
| CENPI | 0.0003 | −1.415 | −5.075 |
| SETD2 | 0.0001 | −1.301 | −5.069 |
| TWISTNB | 0.0007 | −1.601 | −5.056 |
| DNA2 | 0.0001 | −1.312 | −5.055 |
| MMS22L | 0.0008 | −1.639 | −5.053 |
| FOXK1 | 0.0001 | −1.301 | −5.050 |
| PLK4 | 0.0001 | −1.310 | −5.044 |
| ARGLU1 | 0.0002 | −1.329 | −5.007 |
| POLA2 | 0.0001 | −1.298 | −4.988 |
| PAFAH1B1 | 0.0006 | −1.542 | −4.983 |
| WDR61 | 0.0002 | −1.317 | −4.974 |
| PAPD5 | 0.0019 | −1.822 | −4.965 |
| LSM7 | 0.0001 | −1.278 | −4.957 |
| EIF4G2 | 0.0001 | −1.275 | −4.941 |
| CCT7 | 0.0060 | −2.215 | −4.920 |
| SETDB1 | 0.0002 | −1.287 | −4.901 |
| PES1 | 0.0023 | −1.852 | −4.895 |
| CTCF | 0.0008 | −1.582 | −4.870 |
| USP5 | 0.0002 | −1.280 | −4.844 |
| CENPC | 0.0008 | −1.566 | −4.837 |
| SRP19 | 0.0015 | −1.718 | −4.836 |
| SMU1 | 0.0145 | −2.586 | −4.753 |
| STRAP | 0.0001 | −1.241 | −4.752 |
| METTL3 | 0.0015 | −1.683 | −4.746 |
| C7orf26 | 0.0002 | −1.274 | −4.740 |
| FNBP4 | 0.0011 | −1.600 | −4.721 |
| PAK1IP1 | 0.0002 | −1.280 | −4.712 |
| WDR18 | 0.0005 | −1.436 | −4.702 |
| MKI67IP | 0.0006 | −1.457 | −4.702 |
| RBM26 | 0.0001 | −1.206 | −4.684 |
| LSM2 | 0.0028 | −1.836 | −4.679 |
| DCTN2 | 0.0001 | −1.204 | −4.670 |
| DDX23 | 0.0028 | −1.821 | −4.651 |
| COG8 | 0.0002 | −1.289 | −4.647 |
| API5 | 0.0002 | −1.261 | −4.641 |
| NOL11 | 0.0001 | −1.209 | −4.638 |
| GLRX3 | 0.0055 | −2.044 | −4.623 |
| EXOSC9 | 0.0006 | −1.432 | −4.620 |
| MTOR | 0.0003 | −1.285 | −4.613 |
| CDK7 | 0.0002 | −1.232 | −4.571 |
| SEC61G | 0.0032 | −1.836 | −4.570 |
| CHERP | 0.0002 | −1.266 | −4.569 |
| LDLR | 0.0002 | −1.251 | −4.569 |
| GTPBP4 | 0.0001 | −1.173 | −4.539 |
| NGF | 0.0021 | −1.686 | −4.523 |
| TPX2 | 0.0031 | −1.805 | −4.517 |
| CNBP | 0.0002 | −1.198 | −4.504 |
| DPH1 | 0.0002 | −1.237 | −4.497 |
| POLA1 | 0.0005 | −1.361 | −4.488 |
| RCC1 | 0.0001 | −1.166 | −4.477 |
| METTL14 | 0.0017 | −1.612 | −4.450 |
| YTHDF2 | 0.0001 | −1.151 | −4.426 |
| DONSON | 0.0066 | −2.030 | −4.421 |
| NCBP2 | 0.0050 | −1.917 | −4.413 |
| EGLN1 | 0.0003 | −1.242 | −4.405 |
| RPL38 | 0.0008 | −1.431 | −4.397 |
| POLD1 | 0.0021 | −1.639 | −4.392 |
| AHCTF1 | 0.0019 | −1.608 | −4.389 |
| PRPF3 | 0.0009 | −1.442 | −4.372 |
| TCEB3 | 0.0003 | −1.227 | −4.337 |
| SNRPG | 0.0097 | −2.154 | −4.333 |
| DDX24 | 0.0019 | −1.592 | −4.327 |
| SAMM50 | 0.0001 | −1.119 | −4.305 |
| GINS3 | 0.0015 | −1.524 | −4.304 |
| PABPC1 | 0.0002 | −1.140 | −4.291 |
| ATP6V1A | 0.0001 | −1.108 | −4.260 |
| BOD1L1 | 0.0002 | −1.165 | −4.244 |
| KIF18A | 0.0002 | −1.149 | −4.234 |
| ADSL | 0.0002 | −1.149 | −4.227 |
| CCT3 | 0.0039 | −1.750 | −4.226 |
| TIPARP | 0.0002 | −1.123 | −4.221 |
| YPEL5 | 0.0002 | −1.114 | −4.205 |
| TUBGCP3 | 0.0007 | −1.322 | −4.204 |
| NOC3L | 0.0002 | −1.143 | −4.184 |
| WDR5 | 0.0007 | −1.308 | −4.164 |
| WDR43 | 0.0002 | −1.121 | −4.157 |
| AIDA | 0.0002 | −1.136 | −4.155 |
| C11orf73 | 0.0001 | −1.078 | −4.149 |
| BRCA1 | 0.0015 | −1.463 | −4.140 |
| ATP6V1C1 | 0.0001 | −1.072 | −4.131 |
| ATP6V1E1 | 0.0004 | −1.222 | −4.116 |
| C9orf114 | 0.0003 | −1.179 | −4.108 |
| XRCC6 | 0.0040 | −1.708 | −4.099 |
| SF3A2 | 0.0028 | −1.609 | −4.097 |
| MAD2L1BP | 0.0002 | −1.086 | −4.073 |
| WDR55 | 0.0014 | −1.428 | −4.059 |
| HSP90AA1 | 0.0002 | −1.108 | −4.052 |
| POLE2 | 0.0034 | −1.641 | −4.046 |
| CCDC86 | 0.0006 | −1.262 | −4.032 |
| SKA3 | 0.0031 | −1.604 | −4.032 |
| RPL39 | 0.0010 | −1.348 | −4.031 |
| U2SURP | 0.0002 | −1.071 | −4.027 |
| TOP2A | 0.0007 | −1.270 | −4.026 |
| SNRPE | 0.0002 | −1.084 | −4.011 |
| PDCD5 | 0.0002 | −1.086 | −4.008 |
| RPL15 | 0.0029 | −1.569 | −3.987 |
| IK | 0.0007 | −1.244 | −3.960 |
| WNK1 | 0.0004 | −1.176 | −3.945 |
| EXOSC4 | 0.0043 | −1.669 | −3.943 |
| SARNP | 0.0002 | −1.030 | −3.936 |
| UXS1 | 0.0024 | −1.497 | −3.932 |
| SMNDC1 | 0.0002 | −1.083 | −3.922 |
| PFAS | 0.0002 | −1.065 | −3.858 |
| EXOSC2 | 0.0040 | −1.600 | −3.841 |
| KIAA0556 | 0.0002 | −1.017 | −3.814 |
| ZMAT5 | 0.0003 | −1.064 | −3.813 |
| PGD | 0.0035 | −1.549 | −3.796 |
| FAM208A | 0.0004 | −1.109 | −3.781 |
| EBNA1BP2 | 0.0021 | −1.409 | −3.768 |
| CENPP | 0.0026 | −1.461 | −3.768 |
| HYDIN | 0.0005 | −1.149 | −3.756 |
| SNRNP200 | 0.0017 | −1.360 | −3.755 |
| PPP4R2 | 0.0005 | −1.145 | −3.745 |
| RFC1 | 0.0008 | −1.196 | −3.723 |
| POLR3H | 0.0002 | −0.973 | −3.718 |
| MSL3 | 0.0002 | −1.012 | −3.710 |
| PAIP1 | 0.0003 | −1.071 | −3.704 |
| UTP15 | 0.0042 | −1.549 | −3.683 |
| TUBGCP4 | 0.0005 | −1.104 | −3.674 |
| DDX54 | 0.0019 | −1.343 | −3.668 |
| CDK12 | 0.0002 | −0.957 | −3.659 |
| GAR1 | 0.0001 | −0.954 | −3.649 |
| TGS1 | 0.0013 | −1.256 | −3.646 |
| PALB2 | 0.0002 | −0.955 | −3.632 |
| RPL36 | 0.0020 | −1.344 | −3.615 |
| MIS18BP1 | 0.0002 | −0.961 | −3.604 |
| NUF2 | 0.0004 | −1.063 | −3.604 |
| TRIP13 | 0.0004 | −1.062 | −3.600 |
| SPC24 | 0.0028 | −1.409 | −3.595 |
| ZC3H7A | 0.0002 | −0.976 | −3.595 |
| MAT2A | 0.0004 | −1.043 | −3.589 |
| TRAIP | 0.0002 | −0.938 | −3.550 |
| NOP10 | 0.0010 | −1.186 | −3.541 |
| NUTM1 | 0.0010 | −1.186 | −3.541 |
| TAF1B | 0.0017 | −1.271 | −3.534 |
| DSCC1 | 0.0015 | −1.237 | −3.510 |
| DYNLL1 | 0.0002 | −0.926 | −3.503 |
| POLN | 0.0002 | −0.921 | −3.481 |
| NAA38 | 0.0030 | −1.374 | −3.458 |
| DARS | 0.0075 | −1.611 | −3.425 |
| PSPH | 0.0014 | −1.202 | −3.419 |
| EXOSC7 | 0.0055 | −1.511 | −3.413 |
| RAD17 | 0.0009 | −1.112 | −3.392 |
| SEC61B | 0.0002 | −0.904 | −3.391 |
| CASD1 | 0.0158 | −1.876 | −3.379 |
| TRIM56 | 0.0002 | −0.887 | −3.376 |
| RBBP8 | 0.0006 | −1.043 | −3.368 |
| PGAP2 | 0.0005 | −1.024 | −3.359 |
| POLR3C | 0.0014 | −1.174 | −3.355 |
| RBM17 | 0.0008 | −1.076 | −3.330 |
| TRIP12 | 0.0005 | −1.018 | −3.329 |
| RAB5C | 0.0002 | −0.877 | −3.329 |
| HUWE1 | 0.0002 | −0.906 | −3.320 |
| PRPF8 | 0.0042 | −1.397 | −3.319 |
| SUPT5H | 0.0031 | −1.324 | −3.318 |
| GNL3 | 0.0087 | −1.608 | −3.317 |
| TAF1C | 0.0003 | −0.925 | −3.304 |
| LSM3 | 0.0030 | −1.308 | −3.300 |
| MPHOSPH6 | 0.0047 | −1.417 | −3.299 |
| SPDL1 | 0.0030 | −1.303 | −3.294 |
| WDR74 | 0.0030 | −1.300 | −3.278 |
| RBM15 | 0.0225 | −1.988 | −3.274 |
| CTPS1 | 0.0002 | −0.890 | −3.259 |
| CHD2 | 0.0009 | −1.062 | −3.259 |
| TUBGCP6 | 0.0010 | −1.086 | −3.238 |
| POLR3K | 0.0004 | −0.936 | −3.220 |
| PCBP2 | 0.0005 | −0.964 | −3.207 |
| ATP2A2 | 0.0017 | −1.149 | −3.186 |
| HAUS5 | 0.0131 | −1.692 | −3.185 |
| INIP | 0.0038 | −1.313 | −3.182 |
| MRPL13 | 0.0003 | −0.883 | −3.178 |
| TAF5 | 0.0016 | −1.133 | −3.171 |
| CDAN1 | 0.0013 | −1.098 | −3.169 |
| MAU2 | 0.0010 | −1.058 | −3.153 |
| THAP4 | 0.0010 | −1.051 | −3.146 |
| NOL9 | 0.0002 | −0.870 | −3.139 |
| SEC62 | 0.0002 | −0.853 | −3.121 |
| POLR2H | 0.0060 | −1.397 | −3.103 |
| NLE1 | 0.0056 | −1.373 | −3.093 |
| HAUS1 | 0.0039 | −1.278 | −3.077 |
| TYMS | 0.0002 | −0.836 | −3.055 |
| EIF4B | 0.0003 | −0.872 | −3.042 |
| SUGT1 | 0.0004 | −0.887 | −3.035 |
| TAF12 | 0.0020 | −1.120 | −3.030 |
| PRPF38A | 0.0138 | −1.622 | −3.016 |
| ELL | 0.0003 | −0.870 | −3.013 |
| CDK1 | 0.0006 | −0.935 | −3.013 |
| DZANK1 | 0.0019 | −1.099 | −2.996 |
| RNGTT | 0.0005 | −0.908 | −2.993 |
| IPO9 | 0.0003 | −0.843 | −2.992 |
| ZCCHC14 | 0.0188 | −1.724 | −2.975 |
| SNRPD1 | 0.0031 | −1.185 | −2.972 |
| TCEB2 | 0.0393 | −2.107 | −2.962 |
| ACOT12 | 0.0015 | −1.038 | −2.945 |
| UBE2C | 0.0006 | −0.905 | −2.944 |
| TMA16 | 0.0021 | −1.097 | −2.943 |
| SETX | 0.0004 | −0.856 | −2.932 |
| NUP153 | 0.0069 | −1.354 | −2.930 |
| TOP1 | 0.0032 | −1.173 | −2.929 |
| INTS12 | 0.0007 | −0.931 | −2.926 |
| DCTN3 | 0.0008 | −0.944 | −2.925 |
| NOC2L | 0.0051 | −1.277 | −2.924 |
| ADSS | 0.0031 | −1.167 | −2.921 |
| POLR1B | 0.0112 | −1.493 | −2.914 |
| SLC38A1 | 0.0015 | −1.034 | −2.907 |
| TAF6 | 0.0097 | −1.436 | −2.892 |
| SNRNP25 | 0.0005 | −0.868 | −2.883 |
| TAF1 | 0.0026 | −1.115 | −2.880 |
| U2AF1 | 0.0081 | −1.377 | −2.877 |
| SOD1 | 0.0088 | −1.400 | −2.875 |
| TAF9 | 0.0037 | −1.180 | −2.871 |
| ABCB10 | 0.0021 | −1.074 | −2.868 |
| HAUS8 | 0.0005 | −0.858 | −2.865 |
| MPHOSPH8 | 0.0002 | −0.785 | −2.844 |
| COPZ1 | 0.0149 | −1.555 | −2.842 |
| HIST2H3A | 0.0020 | −1.051 | −2.837 |
| HIST2H3C | 0.0020 | −1.051 | −2.837 |
| DNMT1 | 0.0023 | −1.076 | −2.833 |
| METAP2 | 0.0003 | −0.815 | −2.829 |
| ZFP36L2 | 0.0003 | −0.799 | −2.822 |
| CHD8 | 0.0012 | −0.960 | −2.821 |
| DYNLRB1 | 0.0038 | −1.164 | −2.812 |
| SLC39A1 | 0.0002 | −0.765 | −2.811 |
| ZFC3H1 | 0.0004 | −0.825 | −2.805 |
| SKA1 | 0.0092 | −1.371 | −2.794 |
| NDUFA2 | 0.0011 | −0.941 | −2.790 |
| PPRC1 | 0.0131 | −1.478 | −2.785 |
| SRP9 | 0.0169 | −1.568 | −2.779 |
| DGCR8 | 0.0036 | −1.132 | −2.764 |
| NABP1 | 0.0002 | −0.750 | −2.758 |
| C16orf72 | 0.0019 | −1.002 | −2.719 |
| ANAPC4 | 0.0002 | −0.752 | −2.712 |
| EXOC1 | 0.0004 | −0.785 | −2.703 |
| SMARCA5 | 0.0009 | −0.892 | −2.700 |
| PQBP1 | 0.0004 | −0.785 | −2.686 |
| DTYMK | 0.0013 | −0.930 | −2.680 |
| GTF2F1 | 0.0025 | −1.021 | −2.655 |
| LIMCH1 | 0.0094 | −1.293 | −2.622 |
| DCAF4 | 0.0003 | −0.754 | −2.613 |
| RSRC2 | 0.0050 | −1.135 | −2.612 |
| DTL | 0.0307 | −1.723 | −2.606 |
| DYNC1I2 | 0.0014 | −0.915 | −2.605 |
| RRP15 | 0.0101 | −1.300 | −2.595 |
| UBE2I | 0.0344 | −1.772 | −2.594 |
| ZBTB11 | 0.0029 | −1.018 | −2.579 |
| XRCC5 | 0.0004 | −0.761 | −2.561 |
| ADGB | 0.0066 | −1.171 | −2.555 |
| SLC39A9 | 0.0004 | −0.752 | −2.554 |
| KANSL2 | 0.0033 | −1.027 | −2.552 |
| RBM27 | 0.0088 | −1.238 | −2.547 |
| HAUS3 | 0.0017 | −0.921 | −2.544 |
| TAF1D | 0.0024 | −0.971 | −2.542 |
| SKIV2L2 | 0.0006 | −0.791 | −2.541 |
| TUBGCP5 | 0.0029 | −0.998 | −2.537 |
| NDC80 | 0.0187 | −1.466 | −2.534 |
| BOP1 | 0.0121 | −1.314 | −2.519 |
| BANP | 0.0170 | −1.417 | −2.508 |
| FAM171A1 | 0.0005 | −0.756 | −2.506 |
| WDR7 | 0.0005 | −0.754 | −2.498 |
| EXOC3 | 0.0007 | −0.786 | −2.495 |
| ZNF830 | 0.0035 | −1.010 | −2.485 |
| SNAP23 | 0.0015 | −0.875 | −2.478 |
| RBM5 | 0.0055 | −1.095 | −2.477 |
| TAF3 | 0.0052 | −1.085 | −2.475 |
| ZNF259 | 0.0010 | −0.828 | −2.470 |
| TUT1 | 0.0042 | −1.038 | −2.469 |
| POLR3B | 0.0136 | −1.322 | −2.469 |
| NMD3 | 0.0006 | −0.767 | −2.467 |
| RSL24D1 | 0.0034 | −0.995 | −2.455 |
| KIAA0101 | 0.0006 | −0.754 | −2.444 |
| BRCA2 | 0.0269 | −1.549 | −2.432 |
| MTBP | 0.0328 | −1.630 | −2.418 |
| RPL37A | 0.0036 | −0.987 | −2.412 |
| ZFR | 0.0013 | −0.829 | −2.404 |
| CLK3 | 0.0017 | −0.869 | −2.396 |
| SLC44A5 | 0.0029 | −0.944 | −2.389 |
| PPP1R13L | 0.0017 | −0.860 | −2.383 |
| LYPD6 | 0.0034 | −0.964 | −2.381 |
| SYT12 | 0.0009 | −0.786 | −2.376 |
| RBM14 | 0.0089 | −1.153 | −2.366 |
| UVRAG | 0.0016 | −0.845 | −2.364 |
| CHEK1 | 0.0030 | −0.929 | −2.348 |
| FUBP1 | 0.0081 | −1.120 | −2.341 |
| MYBL2 | 0.0009 | −0.771 | −2.340 |
| ISG20L2 | 0.0229 | −1.427 | −2.338 |
| RPL7L1 | 0.0037 | −0.956 | −2.330 |
| DDX27 | 0.0149 | −1.271 | −2.322 |
| UHRF1BP1 | 0.0130 | −1.232 | −2.321 |
| PPHLN1 | 0.0050 | −1.006 | −2.313 |
| GKAP1 | 0.0173 | −1.309 | −2.307 |
| EPRS | 0.0014 | −0.805 | −2.302 |
| RFC5 | 0.0063 | −1.043 | −2.294 |
| ASCC3 | 0.0021 | −0.855 | −2.286 |
| PAPD4 | 0.0012 | −0.781 | −2.285 |
| HIRA | 0.0063 | −1.034 | −2.274 |
| TBCC | 0.0027 | −0.886 | −2.271 |
| HDAC7 | 0.0086 | −1.098 | −2.267 |
| TLN1 | 0.0030 | −0.897 | −2.266 |
| MBIP | 0.0033 | −0.901 | −2.233 |
| C18orf56 | 0.0012 | −0.759 | −2.229 |
| SMC3 | 0.0047 | −0.955 | −2.220 |
| TRAF3IP2 | 0.0016 | −0.792 | −2.219 |
| SPAG17 | 0.0060 | −0.996 | −2.217 |
| CMTR1 | 0.0189 | −1.285 | −2.215 |
| ITGAV | 0.0039 | −0.918 | −2.207 |
| NOP16 | 0.0165 | −1.231 | −2.194 |
| CDC45 | 0.0117 | −1.135 | −2.192 |
| MED21 | 0.0026 | −0.846 | −2.181 |
| RANGAP1 | 0.0317 | −1.454 | −2.180 |
| PRPF40A | 0.0028 | −0.851 | −2.174 |
| TIMELESS | 0.0167 | −1.223 | −2.173 |
| EFTUD2 | 0.0153 | −1.195 | −2.168 |
| FBXO5 | 0.0017 | −0.785 | −2.166 |
| EXOC2 | 0.0015 | −0.758 | −2.151 |
| PPP4C | 0.0044 | −0.910 | −2.147 |
| SART1 | 0.0083 | −1.031 | −2.147 |
| RPL13 | 0.0125 | −1.128 | −2.146 |
| TMEM217 | 0.0118 | −1.098 | −2.117 |
| PELP1 | 0.0203 | −1.231 | −2.085 |
| VAPA | 0.0029 | −0.817 | −2.075 |
| WTAP | 0.0043 | −0.878 | −2.074 |
| ALYREF | 0.0227 | −1.247 | −2.048 |
| RPL6 | 0.0093 | −0.996 | −2.022 |
| EXOSC6 | 0.0211 | −1.202 | −2.015 |
| PNKP | 0.0037 | −0.816 | −1.990 |
| ACTR10 | 0.0195 | −1.163 | −1.990 |
| GSTA4 | 0.0193 | −1.158 | −1.985 |
| TINF2 | 0.0031 | −0.786 | −1.966 |
| COPB1 | 0.0156 | −1.085 | −1.962 |
| CD63 | 0.0091 | −0.961 | −1.959 |
| ATP6V1G1 | 0.0099 | −0.968 | −1.942 |
| NKD1 | 0.0263 | −1.222 | −1.932 |
| ZC3HAV1L | 0.0054 | −0.852 | −1.932 |
| BTNL9 | 0.0030 | −0.764 | −1.923 |
| NUP85 | 0.0171 | −1.087 | −1.921 |
| SNAP91 | 0.0039 | −0.797 | −1.920 |
| KCNT1 | 0.0072 | −0.889 | −1.905 |
| RPL23A | 0.0156 | −1.047 | −1.892 |
| ZMYM3 | 0.0033 | −0.762 | −1.891 |
| SNRNP35 | 0.0057 | −0.843 | −1.891 |
| POLR3F | 0.0177 | −1.077 | −1.887 |
| RUVBL1 | 0.0367 | −1.307 | −1.876 |
| TWSG1 | 0.0043 | −0.789 | −1.869 |
| RPL32 | 0.0040 | −0.778 | −1.867 |
| CHTF18 | 0.0211 | −1.104 | −1.851 |
| SNRNP70 | 0.0175 | −1.050 | −1.844 |
| INTS2 | 0.0124 | −0.958 | −1.826 |
| CCNK | 0.0259 | −1.149 | −1.823 |
| SMIM7 | 0.0140 | −0.979 | −1.816 |
| PCNA | 0.0263 | −1.144 | −1.808 |
| COPB2 | 0.0070 | −0.835 | −1.801 |
| SKA2 | 0.0042 | −0.753 | −1.787 |
| KPNB1 | 0.0285 | −1.156 | −1.787 |
| POLR3E | 0.0125 | −0.937 | −1.782 |
| SLC18A3 | 0.0054 | −0.786 | −1.782 |
| DTHD1 | 0.0196 | −1.040 | −1.775 |
| BCKDHA | 0.0056 | −0.787 | −1.774 |
| EIF5 | 0.0122 | −0.924 | −1.768 |
| PLEKHG5 | 0.0130 | −0.938 | −1.768 |
| LAMTOR2 | 0.0197 | −1.036 | −1.767 |
| OSTC | 0.0050 | −0.769 | −1.766 |
| GLO1 | 0.0316 | −1.170 | −1.755 |
| C1QC | 0.0281 | −1.129 | −1.752 |
| CCND3 | 0.0105 | −0.878 | −1.738 |
| SP1 | 0.0144 | −0.943 | −1.735 |
| SASS6 | 0.0189 | −1.005 | −1.733 |
| ITGAM | 0.0130 | −0.917 | −1.730 |
| MCM7 | 0.0423 | −1.241 | −1.704 |
| DHX9 | 0.0257 | −1.069 | −1.700 |
| TRABD | 0.0055 | −0.752 | −1.699 |
| PPFIBP1 | 0.0062 | −0.766 | −1.690 |
| EXOSC3 | 0.0256 | −1.059 | −1.686 |
| UHRF1 | 0.0161 | −0.932 | −1.673 |
| HIST1H3A | 0.0264 | −1.059 | −1.672 |
| MORC2 | 0.0063 | −0.758 | −1.669 |
| DDHD1 | 0.0239 | −1.027 | −1.666 |
| USPL1 | 0.0225 | −1.010 | −1.664 |
| TMEM229B | 0.0138 | −0.893 | −1.661 |
| VWA9 | 0.0062 | −0.751 | −1.658 |
| SLC23A2 | 0.0082 | −0.792 | −1.653 |
| TTC39C | 0.0082 | −0.792 | −1.652 |
| KIF18B | 0.0104 | −0.821 | −1.628 |
| PRPF4 | 0.0164 | −0.912 | −1.628 |
| CINP | 0.0129 | −0.858 | −1.621 |
| DPY19L1 | 0.0302 | −1.064 | −1.617 |
| TMEM210 | 0.0156 | −0.892 | −1.612 |
| EMC1 | 0.0281 | −1.032 | −1.601 |
| SMC2 | 0.0163 | −0.890 | −1.590 |
| C6orf201 | 0.0177 | −0.905 | −1.587 |
| RPL13A | 0.0202 | −0.936 | −1.585 |
| TSC22D2 | 0.0090 | −0.773 | −1.580 |
| AFMID | 0.0100 | −0.790 | −1.580 |
| RPRD2 | 0.0215 | −0.936 | −1.562 |
| PRSS56 | 0.0326 | −1.042 | −1.550 |
| LPPR2 | 0.0439 | −1.139 | −1.547 |
| JOSD2 | 0.0114 | −0.795 | −1.545 |
| CKAP5 | 0.0161 | −0.856 | −1.535 |
| TCEANC2 | 0.0207 | −0.908 | −1.528 |
| SNRNP27 | 0.0188 | −0.881 | −1.521 |
| CEBPZ | 0.0396 | −1.065 | −1.493 |
| SDR42E2 | 0.0425 | −1.084 | −1.487 |
| IRGQ | 0.0358 | −1.017 | −1.471 |
| BTBD10 | 0.0289 | −0.950 | −1.463 |
| DDX51 | 0.0311 | −0.950 | −1.431 |
| DPF2 | 0.0157 | −0.791 | −1.427 |
| CDK13 | 0.0256 | −0.892 | −1.419 |
| MESDC1 | 0.0143 | −0.768 | −1.416 |
| DIS3 | 0.0205 | −0.835 | −1.410 |
| PPP2R1A | 0.0386 | −0.963 | −1.362 |
| C12orf60 | 0.0353 | −0.934 | −1.356 |
| CDC42 | 0.0234 | −0.808 | −1.318 |
| OR7G2 | 0.0216 | −0.790 | −1.315 |
| ITPRIPL1 | 0.0363 | −0.907 | −1.305 |
| CENPN | 0.0219 | −0.779 | −1.292 |
| CARF | 0.0211 | −0.769 | −1.287 |
| BROX | 0.0325 | −0.863 | −1.285 |
| TMEM68 | 0.0351 | −0.881 | −1.281 |
| TUBB8 | 0.0231 | −0.768 | −1.256 |
| DDX5 | 0.0358 | −0.855 | −1.237 |
| MAP2K4 | 0.0490 | −0.927 | −1.213 |
| CDC23 | 0.0449 | −0.891 | −1.201 |
| MAP3K7 | 0.0307 | −0.765 | −1.157 |
| NUP107 | 0.0324 | −0.774 | −1.153 |
| HEBP1 | 0.0314 | −0.757 | −1.138 |
| ETV4 | 0.0321 | −0.752 | −1.123 |
| ADAMTSL3 | 0.0468 | −0.844 | −1.123 |
| STIL | 0.0375 | −0.771 | −1.100 |
| CSNK1A1 | 0.0427 | −0.795 | −1.089 |
| MARCH3 | 0.0484 | −0.817 | −1.075 |
| HDAC6 | 0.0447 | −0.780 | −1.053 |

“Significant genes” are those have a score where DS = PS*-log10(p-value) < −1
“Discrimination score (DS)” is a combined score that weights the significance and the magnitude of the fold change between the top and bottom bins.
Phenotype score (PS) = log2(top/bottom sg counts) < −0.75
Mann-Whitney p-value < 0.05

TABLE 2
See, Table 1 for definitions of DS.

| | | average | | | | |
|---|---|---|---|---|---|---|
| | Mann- | phenotype | | | Example Targeting (or | |
| | Whitney | of | | | related target) Compounds | Related |
| gene | p-value | strongest 3 | DS | Justification | (may be others) | Target |
| SEC61A1 | 0.0001 | −3.699 | −14.620 | Direct drug | Cotransin; Coibamide A; | |
| | | | | target | Mycolactone (and others) | |
| YTHDC1 | 0.0001 | −3.492 | −13.795 | Direct drug | Published binders: | Possible |
| | | | | target | https://doi.org/10.1016/ | PROTAC |
| | | | | | j.ejmcr.2022.100057 | Candidates |
| SAE1 | 0.0001 | −3.041 | −12.000 | Direct drug | TAK-981 | |
| | | | | target | | |
| RRM1 | 0.0005 | −3.622 | −11.807 | Direct drug | TAS1553; gemcitabine; | |
| | | | | target | clofarabine | |
| POLE | 0.0001 | −2.744 | −10.830 | Direct drug | Aphidicolin | |
| | | | | target | | |
| VHL | 0.0003 | −2.916 | −10.178 | Direct drug | VH298 | PROTAC |
| | | | | target | | Possible- |
| | | | | | | current |
| | | | | | | molecule |
| | | | | | | targets VHL- |
| | | | | | | HIF1 |
| | | | | | | interaction |
| RRM2 | 0.0001 | −2.552 | −10.058 | Direct drug | gemcitabine; clofarabine | |
| | | | | target | | |
| EIF4A1 | 0.0001 | −2.485 | −9.802 | Direct drug | eFT226/Zotatifin | |
| | | | | target | | |
| EEF1A1 | #N/A | #N/A | −9.29 | Direct drug | Ternatin-4 & derivatives | |
| | | | | target | | |
| PRPF4B | 0.0001 | −2.203 | −8.572 | Direct drug | benzothiophene & | |
| | | | | target | derivatives ACH-000224 | |
| UMPS | 0.0001 | −1.953 | −7.686 | Direct drug | Alpha- | |
| | | | | target | Phosphoribosylpyrophosphoric | |
| | | | | | Acid | |
| RAD51 | 0.0001 | −1.866 | −7.337 | Direct drug | CYT-0851 | |
| | | | | target | | |
| AURKA | 0.0007 | −2.026 | −6.358 | Direct drug | Alisertib (MLN8237); | |
| | | | | target | MLN8054 | |
| CDC7 | 0.0002 | −1.625 | −6.119 | Direct drug | BMS-863233; TAK-931 | |
| | | | | target | | |
| KIF11 | 0.0002 | −1.536 | −5.578 | Direct drug | ARRY-520, AZD-4877, | |
| | | | | target | Ispinesib, LY2523355, SB- | |
| | | | | | 743921 | |
| PGGT1B | 0.0001 | −1.374 | −5.287 | Direct drug | L-778123; GGTI-298; L- | |
| | | | | target | 745631 | |
| MBNL1 | 0.0002 | −1.335 | −5.099 | Direct drug | Published inhibitor of RNA | |
| | | | | target | binding: | |
| | | | | | https://doi.org/10.1038/ | |
| | | | | | ncomms3044 | |
| PLK4 | 0.0001 | −1.310 | −5.044 | Direct drug | CFI-400945 | |
| | | | | target | | |
| MTOR | 0.0003 | −1.285 | −4.613 | Direct drug | Rapalogs (Everolimus, | |
| | | | | target | temsirolimus, sirolimus); | |
| | | | | | ATP competive | |
| | | | | | (Torin/PP242/Ku- | |
| | | | | | 0063794/WYE-354); | |
| | | | | | RapaLink-1 | |
| CDK7 | 0.0002 | −1.232 | −4.571 | Direct drug | LDC4297 (LDC044297); | |
| | | | | target | THZ1; THZ2; YKL-5-124; | |
| | | | | | LY3405105 | |
| NGF | 0.0021 | −1.686 | −4.523 | Direct drug | Fasinumab; Tanezumab | |
| | | | | target | | |
| POLA1 | 0.0005 | −1.361 | −4.488 | Direct drug | HO-221 | |
| | | | | target | | |
| EGLN1 | 0.0003 | −1.242 | −4.405 | Direct drug | FG-4592 | |
| | | | | target | | |
| POLD1 | 0.0021 | −1.639 | −4.392 | Direct drug | CMX-001; DFP-14927 | |
| | | | | target | | |
| WDR5 | 0.0007 | −1.308 | −4.164 | Direct drug | OICR-9429 | |
| | | | | target | | |
| BRCA1 | 0.0015 | −1.463 | −4.140 | Direct drug | BRCA1-IN-1 | |
| | | | | target | | |
| HSP90AA1 | 0.0002 | −1.108 | −4.052 | Direct drug | Amlexanox; Cromoglicate; | |
| | | | | target | BIIB-021; Efungumab; KW- | |
| | | | | | 2478; NVP-AUY922; | |
| | | | | | Tanespimycin; VER 50589 | |
| TOP2A | 0.0007 | −1.270 | −4.026 | Direct drug | Camsirubicin; NK314; | |
| | | | | target | Annamycin | |
| PGD | 0.0035 | −1.549 | −3.796 | Direct drug | GHRP-2 | |
| | | | | target | | |
| TYMS | 0.0002 | −0.836 | −3.055 | Direct drug | Plevitrexed (R)-isomer; | |
| | | | | target | NUC-3373 | |
| CDK1 | 0.0006 | −0.935 | −3.013 | Direct drug | Ro-3306; R547 | |
| | | | | target | | |
| TOP1 | 0.0032 | −1.173 | −2.929 | Direct drug | Irinotecan; Topetecan; | |
| | | | | target | Camptothecin (NSC- | |
| | | | | | 100880); Genz-644282 | |
| SOD1 | 0.0088 | −1.400 | −2.875 | Direct drug | BIIB067; Coprexa; ATN-224 | |
| | | | | target | | |
| DNMT1 | 0.0023 | −1.076 | −2.833 | Direct drug | RX-3117; Palifosfamide; | |
| | | | | target | | |
| METAP2 | 0.0003 | −0.815 | −2.829 | Direct drug | Beloranib; ZGN-1061; | |
| | | | | target | M8891 | |
| CLK3 | 0.0017 | −0.869 | −2.396 | Direct drug | KH-CB19 | |
| | | | | target | | |
| CHEK1 | 0.0030 | −0.929 | −2.348 | Direct drug | LY2603618; LY2606368; | |
| | | | | target | SCH-900776; GDC-0575 | |
| | | | | | (ARRY-575) | |
| HDAC7 | 0.0086 | −1.098 | −2.267 | Direct drug | TMP195 (TFMO 2); TMP269 | |
| | | | | target | | |
| ITGAV | 0.0039 | −0.918 | −2.207 | Direct drug | Abituzumab; IMGN-388; | |
| | | | | target | Pracinostat (SB939) | |
| GLO1 | 0.0316 | −1.170 | −1.755 | Direct drug | BAICALEIN; NARINGENIN | |
| | | | | target | | |
| PRPF4 | 0.0164 | −0.912 | −1.628 | Direct drug | benzothiophene & | |
| | | | | target | derivatives ACH-000224 | |
| CDK13 | 0.0256 | −0.892 | −1.419 | Direct drug | THZ531; ZSQ836 | |
| | | | | target | | |
| MAP2K4 | 0.0490 | −0.927 | −1.213 | Direct drug | HWY336; BSJ-04-122; | |
| | | | | target | Cmpds published in | |
| | | | | | https://doi.org/10.1021/ | |
| | | | | | acsmedchemlett.1c00376 | |
| MAP3K7 | 0.0307 | −0.765 | −1.157 | Direct drug | AZ-TAK1 | |
| | | | | target | | |
| CSNK1A1 | 0.0427 | −0.795 | −1.089 | Direct drug | BTX-A51; D-4476; IC261 | |
| | | | | target | | |
| HDAC6 | 0.0447 | −0.780 | −1.053 | Direct drug | Citarinostat; KA2507 | |
| | | | | target | | |
| EIF4H | 0.0001 | −2.071 | −8.169 | Closely | eFT226/Zotatifin | Co-factor of |
| | | | | related to | | eIF4A1 |
| | | | | known drug | | |
| | | | | target | | |
| UBA2 | 0.0002 | −1.658 | −6.191 | Closely | MLN7243 (aka TAK-243) | UBA1 |
| | | | | related to | | |
| | | | | known drug | | |
| | | | | target | | |
| SENP6 | 0.0003 | −1.491 | −5.256 | Closely | VEA260; VEA499; VEA561; | SENP1/2 |
| | | | | related to | Triptolide; others | |
| | | | | known drug | | |
| | | | | target | | |
| LDLR | 0.0002 | −1.251 | −4.569 | Closely | Alirocumab; SPC5001 | LDL; PCSK9 |
| | | | | related to | | inhibitors |
| | | | | known drug | | |
| | | | | target | | |
| EIF4B | #N/A | #N/A | −3.04 | Closely | eFT226/Zotatifin | Co-factor of |
| | | | | related to | | eIF4A1 |
| | | | | known drug | | |
| | | | | target | | |
| SNAP23 | 0.0015 | −0.875 | −2.478 | Closely | Botulinum Toxin Type A | SNAP25 |
| | | | | related to | | |
| | | | | known drug | | |
| | | | | target | | |
| GSTA4 | 0.0193 | −1.158 | −1.985 | Closely | Ezatiostat | GSTP1 |
| | | | | related to | | |
| | | | | known drug | | |
| | | | | target | | |
| SLC18A3 | 0.0054 | −0.786 | −1.782 | Closely | Ingrezza; Alseroxylon; | SLC18A2 |
| | | | | related to | Reserpine | |
| | | | | known drug | | |
| | | | | target | | |
| CCND3 | 0.0105 | −0.878 | −1.738 | Closely | ABT-263; Briciclib | Related to |
| | | | | related to | | CCND1 |
| | | | | known drug | | |
| | | | | target | | |
| CDC42 | 0.0234 | −0.808 | −1.318 | Closely | EHT-1864 | RAC1 |
| | | | | related to | | |
| | | | | known drug | | |
| | | | | target | | |
| TUBB8 | 0.0231 | −0.768 | −1.256 | Closely | Vindesine; Vincristine; | TUBB |
| | | | | related to | Taxol; Mebendazole | |
| | | | | known drug | | |
| | | | | target | | |

What is claimed is:

1. A bidirectional reporter nucleic acid construct for identifying regulators of oncogene or tumor suppressor gene translation, the reporter system comprising: (a) a first promoter operably linked to a first polynucleotide comprising a nucleic acid sequence encoding a first reporter molecule; and (b) a second promoter, different from the first promoter, operably linked to a second polynucleotide comprising a nucleic acid sequence encoding a second reporter molecule, wherein the second promoter and second polynucleotide are oriented in the opposite orientation relative to that of the first promoter and first polynucleotide, and wherein the second polynucleotide comprises a 5′ untranslated region from an oncogene or tumor suppressor gene at the 5′ end of the nucleic acid sequence encoding the second reporter molecule.

2. The bidirectional reporter nucleic acid construct claim 1, wherein the second reporter molecule is a destabilized green fluorescent protein or firefly luciferase.

3. The bidirectional reporter nucleic acid construct of claim 1 or 2, wherein the second promoter is an elongation factor-1α (EF1α) promoter.

4. The bidirectional reporter nucleic acid construct of claim 1, 2, or 3, where in the first promoter is a human phosphoglycerate kinase promoter.

5. The bidirectional reporter nucleic acid construct of claims 1-4, wherein the first reporter molecule is a red fluorescent protein.

6. The bidirectional reporter nucleic acid construct of claims 1-4, wherein the first reporter molecule is destabilized mCherry or Renilla luciferase.

7. The bidirectional reporter nucleic acid construct of any one of claims 1-6, wherein the oncogene is a RAS family member, a Myc family member, a receptor tyrosine kinase, a cell cycle polypeptide, a kinase, or a hormone receptor; or the tumor suppressor gene is a cell cycle inhibitor, Phosphatase and Tensin Homolog deleted on Chromosome 10 (PTEN) polypeptide, a polypeptide involved in DNA damage response, a Wnt signaling protein, or a p53 family member.

8. A host cell comprising a bidirectional reporter system of any one of claims 1-7.

9. The host cell of claim 8, wherein the host cell is a cancer cell.

10. The host cell of claim 8 or 9, further comprising (i) CRISPRi components comprising a polynucleotide encoding a catalytically inactivated Cas protein linked to a polynucleotide encoding a transcription repressor protein operably linked to an inducible promoter, and
(ii) a nucleic acid construct from a whole genome gRNA library, wherein the nucleic acid construct encodes a gRNA that targets a gene.

11. The host cell of claim 10, wherein the catalytically inactivated Cas protein is dCas9, the transcription repressor is a Kruppel-associated box (KRAB) protein; and
the inducible promoter is a tetracycline promoter.

12. A plurality of cells comprising a population in which each cell contains a reporter construct of any one of claims 1-7.

13. A plurality of host cells comprising a population of host cells in which each host cell comprises a bidirectional reporter nucleic acid construct of any one of claims 1-7; CRISPRi components comprising a polynucleotide encoding a catalytically inactivated Cas protein linked to a polynucleotide encoding a transcription repressor protein operably linked to an inducible promoter, and a nucleic acid construct from a whole genome gRNA library, wherein the nucleic acid encodes a gRNA that targets a gene; and
wherein the population of host cells comprises (i) individual host cells that each comprise a gRNA that target different genes; and (ii) at least two individual host cells that each contain a gRNA that targets the same gene, but differ in sequence from one another.

14. The plurality of host cells of claim 13, wherein the catalytically inactivated Cas protein is dCas9, the transcription repressor is a Kruppel-associated box (KRAB) protein; and the inducible promoter is a tetracycline promoter.

15. A method of identifying a regulator of oncogene or tumor suppressor translation, the method comprising:
(a) inducing expression of the catalytically inactivated Cas protein linked to a transcription repressor protein in the population of host cells of claim 13 or 14;
(b) detecting signal from the first reporter molecule and signal from the second reporter molecule in individual cells of the population of host cells to determine the ratio of signal from the first reporter molecule to signal from the second reporter molecule;
(c) identifying (i) a subpopulation of cells exhibiting the highest ratio of signal from the first reporter molecule to signal from the second reporter molecule and (ii) a 9 subpopulation of cells exhibiting the lowest ratio of signal from the first reporter molecule to signal from the second reporter molecule;
(d) isolating genomic DNA from each of subpopulation of (c);
(e) amplifying sgRNA sequences in DNA from each subpopulation of (c) to obtain nucleic acid preparations;
(f) sequencing nucleic acid preparations of (e) to identify target genes of the sgRNA sequences present in each subpopulation of (c);
(g) quantifying the sgRNAs for each target gene present in subpopulation (c) (i) relative to subpopulation (c) (ii) to identify genes that exhibit at least a two-fold statistically significant change in level in subpopulation (c) (i) compared to subpopulation 19 (c) (ii); and
(h) selecting candidate genes that are enriched in subpopulation (c) (i) as candidate translation repressors and selecting candidates genes that are enriched in subpopulation (c) (ii) as candidate translational activators.

16. The method of claim 15, wherein (c) comprise identifying cells having at least a 2-fold change in expression of the first reporter molecule relative to the average phenotype score; and/or (c) comprises identifying cells having least a 2-fold change in expression of the second reporter molecule relative to the average phenotype score.

17. The method of claim 15 or 16, wherein detecting step (b) comprises fluorescence activated cell sorting.

18. The method of claim 15 or 16, wherein the population of cells is distributed to compartments of a microwell plate and detecting step (b) comprises detecting a fluorescent signal or a luminescent signal from one or more cells distributed to a microwell.

19. The method of claim 18, wherein at least about 200 cells are distributed to each compartments of the microwell plate.

20. The method of claim 15 or 16, wherein detecting step (b) comprises microscopy.

21. The method of claim 15 or 16, wherein detecting step (b) comprises detecting a luminescent signal with a microplate reader.

22. A method of analyzing a target gene identified in any one of claims 16-20, the method comprising inhibiting expression of a gene selected in step (h) in a cell and determining the endogenous level of the oncogene or tumor suppressor protein in the cell.

23. The method of claim 22, wherein inhibiting expression comprises introducing an inhibitory RNA into the cell that targets the gene selected in step (h).

24. The method of claim 23, wherein the inhibitory RNA is an shRNA.

25. The method of claim 22, 23, or 24, wherein the cell is a cancer cell.

26. The method of claim 22-25, further comprising determining the endogenous level of the oncogene or tumor suppressor mRNA in the cell.

27. The method of claim 22-25, further comprising polysome profiling to determine whether the distribution of RNA encoding the oncogene or tumor suppressor as highly translated, untranslated, or translated at a low level has changed compared to a control cell in which expression of the target gene is not inhibited.

28. The method of any one of claims 22-27, further comprising determining whether the cell grows in soft agar.

29. A method of inhibiting growth of a cancer cell, the method comprising treating the cancer cell with an inhibitor of expression of an RNA-Binding motif 42 (RBM42) protein.

30. The method of claim 29, wherein the inhibitor is an inhibitory RNA molecule targets an RBM42 nucleic acid sequence to inhibit expression.

31. The method of claim 29, wherein the cancer cell is from an epithelial-type tumor or a hematological cancer.

32. A method of identifying a regulator of oncogene or tumor suppressor translation, the method comprising:
(a) incubating a cell population comprising individual cells comprising a reporter construct of any one of claims 1-7 with a plurality of candidate agents for regulating oncogene or tumor suppressor translation, where individual portions of the cell population are each incubated with a candidate agent or a pool of candidate agents;
(b) detecting signal from the first reporter molecule and signal from the second reporter molecule in individual cells of the portions to determine the ratio of signal from the first reporter molecule to signal form the second reporter molecule;
(c) identifying (i) a portion of cells exhibiting the highest ratio of signal from the first reporter molecule to signal from the second reporter molecule and (ii) a portion of cells exhibiting the lowest ratio of signal from the first reporter molecule to signal from the second reporter molecule; and
(d) selecting candidate genes that are enriched in portion (c) (i) as candidate translation repressors and selecting candidates genes that are enriched in portion (c) (ii) as candidate translational activators.

33. The method of claim 32, wherein the agent is a small molecule.

34. The method of claim 32, or 33, wherein the cell population comprises cancer cells.

35. A method of evaluating a candidate agent selected by the method of any one of claims 32-34, comprising determining the endogenous level of the oncogene or tumor suppressor mRNA and/or protein in a cell treated with the candidate agent compared to untreated control.

36. The method of claim 35, further comprising determining the amount of protein produced from mRNA transcripts in cells treated with the candidate agent compared to untreated control cells.

37. The method of claim 35 or 36, further comprising evaluating the ability of cells treated with the candidate agent to grow in soft agar and/or form two-dimensional colonies compared to untreated control cells; and/or evaluating cellular proliferation rates or apoptosis compared to untreated control cells.


SEQUENCE LISTING (3 sequences)

SEQ ID NO: 1 - DNA, 408 bp, Homo sapiens
  source 1..408: mol_type = genomic DNA
aactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacg

SEQ ID NO: 2 - DNA, 846 bp, synthetic construct
  source 1..846: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtag

SEQ ID NO: 3 - DNA, 837 bp, synthetic construct
  source 1..837: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgtag